(19) United States
(12) Patent Application Publication
(10) Pub. No.: US 2025/0305034 A1
(43) Pub. Date: Oct. 2, 2025

(54) NANOSTRUCTURES FOR MODULATION OF ANALYTE CONFORMATION

(71) Applicant: Nautilus Subsidiary, Inc., Seattle, WA (US)

(72) Inventors: Terren R. CHANG, Redwood City, CA (US); Maureen NEWMAN, Redwood City, CA (US); Michael Augusto DARCY, Fremont, CA (US); Kara JUNEAU, San Carlos, CA (US); Maryam JOUZI, Hayward, CA (US); Aimee SANFORD, San Mateo, CA (US); Tanvir SAINI, San Mateo, CA (US); Martin HUBER, Menlo Park, CA (US)

(21) Appl. No.: 19/091,119

(22) Filed: Mar. 26, 2025

Related U.S. Application Data
(60) Provisional application No. 63/572,729, filed on Apr. 1, 2024.

(51) Int. Cl. C12Q 1/6834; B82Y 15/00; C12Q 1/6825

(52) U.S. Cl. CPC C12Q 1/6834; C12Q 1/6825; B82Y 15/00

(57) ABSTRACT

Systems and methods for immobilizing macromolecules on solid supports are described. The systems and methods facilitate attachment of macromolecules at multiple attachment points. Structural conformations of attached macromolecules may be altered by disclosed systems and methods. Macromolecules may be provided in useful structural conformations for interrogation by detectable binding reagents.

### RELATED APPLICATIONS

This application claims priority to U.S. Provisional Patent Application No. 63/572,729, titled “Nanostructures for Modulation of Analyte Conformation”, filed on Apr. 1, 2024, the full disclosure of which is hereby incorporated herein by reference in its entirety for all purposes.

### BACKGROUND

Macromolecules, including synthetic and biological polymers can adopt three-dimensional conformations that are determined in part by the primary molecular structures of the macromolecules (i.e., the spatial arrangement of atoms and the types of bonds that connect the atoms to form the macromolecule). For example, the amino acid sequence of a protein determines in part the complex three-dimensional conformation of the protein. Likewise, single-stranded nucleic acids can adopt two- or three-dimensional conformations due to hybridization of internally self-complementary nucleotide sequences. Branched or dendrimeric polymers, including modified biopolymers, can also adapt complex three-dimensional conformations.

The conformation of macromolecules may be further influenced by the environment within which the macromolecules are disposed. Within a fluidic environment, chemical variables, including solvent composition, macromolecules concentration, fluidic pH, and fluidic ionic strength can impact the three-dimensional conformations adopted by the macromolecules. For example, the tertiary structures formed by proteins may change when the pH of a fluidic medium containing the proteins is altered. Accordingly, under particular fluidic conditions a macromolecule may obtain a conformation that sequesters certain residues or regions of the macromolecule from contact with a fluidic medium containing the macromolecule.

### SUMMARY

In an aspect, provided herein is a composition, comprising: a) a particle comprising a first face and a second face, wherein the first face is substantially opposed to the second face, wherein the second face comprises a first attachment site containing a first attachment moiety, and wherein the second face further comprises a second attachment site containing a second attachment moiety, b) a plurality of coupling moieties coupled to the first face, and c) an analyte, wherein the analyte comprises a first complementary attachment moiety and a second complementary attachment moiety, wherein the first complementary attachment moiety is attached to the first attachment moiety, and wherein the second complementary attachment moiety is attached to the second attachment moiety.

In another aspect, provided herein is a method, comprising: a) contacting an analyte to a particle, wherein the particle comprises a first attachment site comprising a first attachment moiety, and a second attachment site comprising a second attachment moiety, b) attaching a first complementary attachment moiety of the analyte to the first attachment moiety of the particle, and attaching a second complementary attachment moiety of the analyte to the second attachment moiety of the particle, and c) coupling the particle to a site of a solid support.

In another aspect, provided herein is a method, comprising: a) contacting a plurality of binding reagents to a solid support, wherein the solid support comprises a plurality of sites, wherein each site comprises a particle, wherein the particle comprises a first attachment site and a second attachment site, and wherein one and only one analyte is attached to the first attachment site and the second attachment site of the particle, b) coupling binding reagents to analytes at sites of the plurality of sites, and c) for each individual site, detecting presence or absence of a signal from a binding reagent of the plurality of binding reagents.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A, 1B, and 1C depict various conformations of a macromolecule between an extended conformation and a compacted or globular conformation, in accordance with some embodiments.

FIG. 2 illustrates the effect of attachment site separation on the conformation of a macromolecule, in accordance with some embodiments.

FIG. 3 shows differences in macromolecule size based upon unoccupied attachment sites, in accordance with some embodiments.

FIG. 4 displays a method of determining macromolecule size based upon detection of signals from detectable labels at unoccupied attachment sites, in accordance with some embodiments.

FIGS. 5A, 5B, 5C, 5D, 5E, and 5F depict steps of methods of providing a macromolecule in an extended conformation utilizing an analyte-binding group, in accordance with some embodiments.

FIGS. 6A, 6B, and 6C illustrate attachment of a macromolecule containing a plurality of attachment moieties, in accordance with some embodiments.

FIGS. 7A and 7B show steps of a method of detecting a binding reagent bound to a macromolecule, in accordance with some embodiments.

FIGS. 8A, 8B, 8C, and 8D display attachment of a macromolecule to an attachment site utilizing an analyte-binding group and an additional attachment moiety, in accordance with some embodiments.

FIGS. 9A, 9B, and 9C depict a method of altering a conformation of a macromolecule by changing attachment sites attached to the macromolecule, in accordance with some embodiments.

FIGS. 10A, 10B, and 10C depict an example of using TCO and mTz to retain a macromolecule in a denatured state.

FIGS. 11A and 11B depict an example of unfolding a macromolecule via tackboard pinning.

FIGS. 12A, 12B, and 12C depict an example of attaching landing sites and tackboards upon a surface.

FIGS. 13A and 13B depict an example of unfolding a macromolecule at different lengths.

FIG. 14 depicts a flow diagram for probing a macromolecule with an unfolded spatial conformation at different lengths.

FIG. 15 depicts a system for performing the techniques described herein.

### DETAILED DESCRIPTION

Within a fluid medium, a macromolecule may assume a spatial conformation or morphology. The particular conformation assumed by the macromolecule may be, at least in part, a function of the molecular structure of the macromolecule, as well as the composition and fluidic properties of the fluid medium. For example, a hydrophobic or non-polar polymer within an aqueous medium may inherently assume a globular morphology to minimize the amount of the polymer contacted to the aqueous medium. In another example, a protein within an aqueous medium may form complex secondary and/or tertiary structures that orient certain hydrophilic amino acid sidechains toward contact with the aqueous medium while sequestering certain hydrophobic amino acid sidechains away from contact with the aqueous medium. For any given morphology or conformation of a macromolecule, some amount of spatial variation may naturally occur due to natural vibrations and rotations of intramolecular bonds as well as collisions with surrounding molecules.

As spatial conformation or morphology of a macromolecule is influenced by the fluid environment surrounding the macromolecule, changes in the fluid environment, such as changes in fluid composition, pH, and/or ionic strength, can provoke changes in conformation or morphology of the macromolecule. A change in fluid environment may induce a change in conformation or morphology of a macromolecule, including formation of one or more secondary and/or tertiary structures, disruption of one or more secondary and/or tertiary structures, or rearrangement of a first secondary or tertiary structure into a second secondary or tertiary structure. Changes in conformation or morphology of a certain macromolecules, such as biomolecules (e.g., proteins, nucleic acids, polysaccharides, and complexes thereof) may be physically or biologically relevant for at least one of the reasons that: 1) macromolecules may naturally vary between two or more conformations or morphologies with associated properties or activities associated with each of the two or more conformations or morphologies, 2) disruption of a conformation or morphology of a macromolecule may disrupt an activity of the macromolecule or alter a physical property, and 3) disruption of a conformation or morphology of a macromolecule may provide accessibility to residues or moieties sequestered within disrupted structures of the macromolecule.

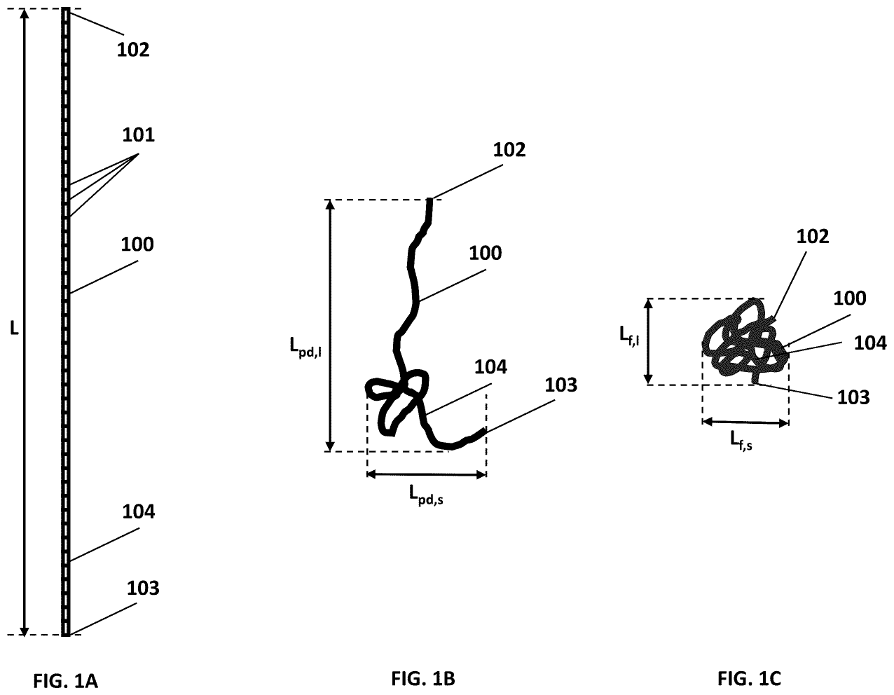

Assays that interrogate macromolecules may depend in part upon the fluid accessibility of certain structures, moieties, or epitopes of the macromolecules. FIGS. 1A-1C illustrate aspects of structural accessibility for a macromolecule with a polymeric chain structure. FIG. 1A depicts a fully extended linear conformation having a total contour length, L, of a polymeric chain 100 comprising a concatenated sequence of monomers, residues, or other moiety 101 (e.g., repeating sequences of linked monomers). The polymeric chain has a first terminal monomer, residue, or moiety 102 and a second terminal monomer, residues, or moiety 103. In the absence of any three-dimensional structuring or folding of the polymeric chain 100, all monomers, residues, or moieties 101 of the polymeric chain, including a non-terminal monomer, residue, or moiety 104 is accessible to a fluid medium surrounding the polymeric chain 100. FIG. 1B depicts a partially-denatured conformation of the polymeric chain 100, in which some monomers, residues, or moieties 101 have folded into a slightly more compact three-dimensional structure. Portions of the polymeric chain 100 nearer to the first terminal monomer, residue, or moiety 102 and the second terminal monomer, residue, or moiety 103 retain an extended or non-folded conformation. The non-terminal monomer, residue, or moiety 104 is located near the folded structure, so contact between the non-terminal monomer, residue, or moiety 104 and the fluid medium may be partially- or fully-occluded. In the conformation of FIG. 1B, the structure of the polymeric chain has a maximum characteristic dimension of L_{pd,1}, and a shorter characteristic dimension of L_{pd,s}, each of which is shorter than the contour length L of the fully extended conformation of FIG. 1A. FIG. 1C depicts a fully-folded or globular conformation of the polymeric chain 100, in which many monomers, residues, or moieties 101 are folded into the three-dimensional conformation of the polymeric chain 100. In this conformation, terminal monomers, residues, or moieties 102 and 103 and non-terminal monomer, residue, or moiety 104 may be exposed at the edge of the three-dimensional structure, or sequestered into an internal portion of the three-dimensional structure. In the conformation of FIG. 1B, the structure of the polymeric chain has a maximum characteristic dimension of L_{f,l}, and a shorter characteristic dimension of L_{f,s}, each of which is shorter than the contour length L of the fully extended conformation of FIG. 1A, and each of which may be shorter than corresponding L_{pd,1} and L_{pd,s} of the conformation of FIG. 1B.

Under assay conditions, macromolecules may assume conformations or morphologies that sequester relevant structures, moieties, or epitopes within fluid-inaccessible portions of the macromolecules, thereby limiting the effectiveness of the assay. This may be true with, for example, affinity agent-based binding assays, where epitopes for affinity agents may be buried within internal structures of assayed macromolecules, thereby inhibiting binding of the affinity agents to the epitope. In some cases, fluidic medium conditions that result in a buried epitope becoming exposed to the fluidic medium may also inhibit the structure and/or function of the affinity agents that are supposed to bind to the epitopes.

Provided herein are systems and methods for manipulating the conformations of macromolecules. The systems and methods may facilitate the display of macromolecules on solid supports. Macromolecules may be displayed in extended conformations that facilitate fluid contact with significant portions of the macromolecule. Further, the systems and methods for displaying the macromolecules may inhibit changes in macromolecular conformation during changes in assay conditions.

### Definitions

Terms used herein will be understood to take on their ordinary meaning in the relevant art unless specified otherwise. Several terms used herein and their meanings are set forth below.

In some embodiments set forth herein, the terms “affinity reagent” and “affinity agent” can refer synonymously to a molecule or other substance that is capable of specifically or reproducibly binding to an analyte (e.g., protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)2 fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof. In some embodiments set forth herein, the term “antibody” may refer to a protein that binds to an antigen or epitope via at least one complementarity determining region (CDR). An antibody can include all elements of a full-length antibody. However, an antibody need not be full length and functional fragments can be particularly useful for many uses. The term “antibody” as used herein encompasses full length antibodies and functional fragments thereof.

In some embodiments set forth herein, the term “array” may refer to a population of analytes (e.g., proteins) that are associated with unique identifiers such that the analytes can be distinguished from each other. A unique identifier can be, for example, a solid support (e.g., particle or bead), address on a solid support, tag, label (e.g., luminophore), or barcode (e.g., nucleic acid barcode) that is associated with an analyte and that is distinct from other identifiers in the array. Analytes can be associated with unique identifiers by attachment, for example, via covalent bonds or non-covalent bonds (e.g., ionic bond, hydrogen bond, van der Waals forces, electrostatics etc.). An array can include different analytes that are each attached to different unique identifiers. An array can include different unique identifiers that are attached to the same or similar type of analyte. An array can include separate solid supports or separate addresses that each bear a different analyte, wherein the different analytes can be identified according to the locations of the solid supports or addresses.

In some embodiments set forth herein, the term “attached” may refer to the state of two things being joined, fastened, adhered, connected, coupled, or bound to or with each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

In some embodiments set forth herein, the term “attachment moiety” may refer to a moiety that couples a macromolecule to a site of an array. An attachment moiety may be attached to a surface of a solid support. An attachment moiety may be attached to a macromolecule. A macromolecule may be coupled to a site of an array by a binding interaction between an attachment moiety attached to a surface of a solid support and a complementary attachment moiety coupled to a macromolecule.

In some embodiments set forth herein, the term “attachment site,” when used in reference to an array site or a particle attached thereto, may refer to a specific location containing an attachment moiety. An array site can contain one or more attachment sites. For example, a site may contain two or more attachment sites, in which a macromolecule is attached to the array site by binding interactions with attachment moieties at at least two of the two or more attachment sites.

In some embodiments set forth herein, the term “avidity component” may refer to a moiety of a first binding partner that is configured to interact with a moiety of a second binding partner to increase the rate of association between the first and second binding partners and/or to decrease the rate of dissociation the first and second binding partners. The first binding partner can further include a primary epitope moiety that interacts with a primary paratope moiety of the second binding partner, or vice versa. An avidity component can include a polymer, nucleic acid strand, nucleic acid duplex, nucleotide sequence, protein, affinity reagent, secondary epitope, secondary paratope, receptor, ligand or the like. A first avidity component can interact with a second avidity component via reversible binding, for example, via non-covalent binding or reversible covalent binding.

In some embodiments set forth herein, the term “binding reagent” may refer to an affinity agent attached to a detectable label. Accordingly, a binding reagent may be configured to produce a detectable signal that facilitates determining a spatial location of the binding reagent. A binding reagent may comprise two or more affinity agents. A binding reagent may comprise two or more detectable labels. An affinity agent of a binding reagent may be attached to a detectable label by a linking moiety or particle (e.g., a nucleic acid nanoparticle, a polymer nanoparticle).

The term “comprising” is intended herein to be open-ended, including not only the recited elements, but further encompassing any additional elements.

In some embodiments set forth herein, the terms “conformational state” and “conformation,” when used in reference to a molecule or particle, may refer to the shape or proportionate dimensions of the molecule or particle. At the molecular level conformational state can be characterized by the spatial arrangement of a molecule that results from the rotation of its atoms about their bonds. The conformational state of a macromolecule, such as a protein or nucleic acid, can be characterized in terms of secondary structure, tertiary structure, or quaternary structure. Secondary structure of a nucleic acid is the set of interactions between bases of the nucleic acid such as interactions formed by internal complementarity in a single stranded nucleic acid or by complementarity between two strands in a double helix. Tertiary structure of a nucleic acid is the three-dimensional shape of the nucleic acid as defined, for example, by the relative locations of its atoms in three-dimensional space. Quaternary structure of a nucleic acid is the overall shape resulting from interactions between two or more nucleic acids at a higher level than the secondary or tertiary levels. Secondary structure of a protein is the three-dimensional form of local segments of the protein which can be defined, for example, by the pattern of hydrogen bonds between the amino hydrogen and carboxyl oxygen atoms in the peptide backbone or by the regular pattern of backbone dihedral angles in a particular region of the Ramachandran plot for the protein. Tertiary structure of a protein is the three-dimensional shape of a single polypeptide chain backbone including, for example, interactions and bonds of side chains that form domains. Quaternary structure of a protein is the three-dimensional shape and interaction between the amino acids of multiple polypeptide chain backbones. A molecule or particle having a given composition may take on more than one conformational state with or without changes to its composition. For example, a protein having a given amino acid sequence (i.e., protein primary structure) may take on different conformations at the secondary, tertiary or quaternary level, and a nucleic acid having a given nucleotide sequence (i.e., nucleic acid primary structure) may take on different conformations at the secondary, tertiary or quaternary level.

In some embodiments set forth herein, the term “covalent,” when used in reference to a bond between atoms or moieties of a molecule, may refer to bonding due to sharing of a pair of electrons between the two atoms or moieties. Covalent interactions can include reversible and irreversible binding interactions. Covalent interaction can arise due to a chemical reaction between a first reactive moiety and a second reactive moiety, optionally in the presence of a third intermediary or catalytic moiety. Covalent binding interactions can form between two atoms or moieties due to various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can also form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 and 11,505,796, each of which is herein incorporated by reference in its entirety.

In some embodiments set forth herein, the term “docker” may refer to a molecule or moiety that is configured to interact with a tether or that is interacting with a tether. A docker can be a moiety of a substance, object, molecule, solid support, address, particle, or bead. A docker can include a polymer, nucleic acid strand, nucleic acid duplex, nucleotide sequence, protein, affinity reagent, epitope, paratope, receptor, ligand or the like. A docker can interact with a tether via covalent or non-covalent bonding.

In some embodiments set forth herein, the term “each,” when used in reference to a collection of items, may be intended to identify an individual item in the collection but does not necessarily refer to every item in the collection. Exceptions can occur if explicit disclosure or context clearly dictates otherwise.

In some embodiments set forth herein, the term “epitope” may refer to a molecule or part of a molecule, which is recognized by or binds specifically to an affinity reagent or paratope. Epitopes may include amino acid sequences that are sequentially adjacent in the primary structure of a protein, or amino acids that are structurally adjacent in the secondary, tertiary or quaternary structure of a protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

In some embodiments set forth herein, the term “face” may refer to a portion of a molecule, particle, or complex (e.g., a SNAP or a SNAP complex) that contains one or more moieties with substantially similar orientation and/or function. For example, a substantially rectangular or square SNAP may have a coupling face that comprises one or more coupling moieties, with each coupling moiety having a substantially similar orientation to each other coupling moiety (e.g., oriented about 180° from a display moiety that is configured to be coupled to an analyte). In another example, a spherical nanoparticle may have a coupling face comprising a coupled plurality of coupling moieties confined to a hemisphere of the particle (i.e., a plurality of coupling moieties having similar function but differing orientations). In some cases, a face may be defined by an imaginary plane relative to which a moiety or a portion thereof may have a spatial proximity or angular orientation when the plane is contacted with a point or portion of a molecule, particle, or complex. A moiety or a portion thereof may have a spatial separation from an imaginary plane defining a face of a molecule, particle, or complex of no more than about 100 nanometers (nm), 90 nm, 80 nm, 70 nm, 60 nm, 50 nm, 40 nm, 30 nm, 25 nm 20 nm, 15 nm, 10 nm, 9 nm, 8 nm, 7 nm, 6 nm, 5 nm, 4 nm, 3 nm, 2 nm, 1 nm, 0.5 nm, 0.1 nm, or less than 0.1 nm. A moiety or a portion thereof may have an angular orientation relative to a normal vector of an imaginary plane of no more than about 90°, 85°, 80°, 75°, 70°, 65°, 60°, 55°, 50°, 45°, 40°, 35°, 30°, 25°, 20°, 15°, 10°, 5°, 1°, or less than 1°.

In some embodiments set forth herein, the terms “group” and “moiety” may refer intended to be synonymous when used in reference to the structure of a molecule. The terms refer to a component or part of the molecule. The terms do not necessarily denote the relative size of the component or part compared to the rest of the molecule, unless indicated otherwise.

In some embodiments set forth herein, the terms “label” and “detectable label” may refer synonymously to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

In some embodiments set forth herein, the terms “linker” and “linking moiety” may refer synonymously to a moiety that connects two objects to each other. One or both objects can be a molecule, solid support, address, particle or bead. Both objects can be moieties of a molecule, solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, protein, nucleotide, nucleic acid, nucleic acid origami, dendrimer, protein nucleic acid (PNA), polysaccharide, carbon, nitrogen, oxygen, ether, sulfur, or disulfide. A linker can be a bead or particle such as a structured nucleic acid particle.

In some embodiments set forth herein, the term “macromolecule” may refer to a molecule, particle, or complex with a molecular weight of 1 kiloDalton (kDa) or more. Macromolecules can include biomolecules (e.g., polypeptides, nucleic acids, polysaccharides, etc.), polymeric molecules, and nanoparticles or microparticles (organic nanoparticles, organic microparticles, inorganic microparticles, inorganic nanoparticles, etc.). In some cases, a macromolecule of a plurality of macromolecules can comprise an analyte of interest. For example, an analyte of interest may be an analyte separated from, purified from, or otherwise derived from a biological sample (e.g., a tissue sample, a cell, a biological fluid, etc.). In some cases, a macromolecule of a plurality of macromolecules can comprise an anchoring moiety. An anchoring moiety may comprise a particle (e.g., a nucleic acid nanoparticle) that is configured to bind to a surface of an array site, and is further configured to bind an analyte to the array site (optionally occluding contact between the array site and the analyte). In some cases, a macromolecule of a plurality of macromolecules may comprise a binding reagent.

In some embodiments set forth herein, the term “non-covalent,” when used in reference to a bond between atoms or moieties of a molecule, may refer to bonding due a mechanism other than electron pair-sharing between the two atoms or moieties. Non-covalent interaction can arise due to an electrostatic or magnetic interaction between moieties and/or atoms. Non-covalent binding interactions can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent interaction may include hybridization of a first oligonucleotide to a complementary second oligonucleotide. In some cases, a non-covalent interaction may form between a receptor and ligand, such as streptavidin-biotin. Other useful non-covalent interactions can include affinity reagent-target interactions, such as antibody-epitope or aptamer-epitope interactions.

In some embodiments set forth herein, the terms “nucleic acid nanostructure” or “nucleic acid nanoparticle,” may refer synonymously to a single- or multi-chain polynucleotide molecule comprising a compacted three-dimensional structure. The compacted three-dimensional structure can optionally have a characteristic tertiary structure. An exemplary nucleic acid nanostructure is a structured nucleic acid particle (SNAP). A SNAP can be configured to have an increased number of interactions between regions of a polynucleotide strand, less distance between the regions, increased number of bends in the strand, and/or more acute bends in the strand, as compared to the same nucleic acid molecule in a random coil or other non-structured state. Alternatively or additionally, the compacted three-dimensional structure of a nucleic acid nanostructure can optionally have a characteristic quaternary structure. For example, a nucleic acid nanostructure can be configured to have an increased number of interactions between polynucleotide strands or less distance between the strands, as compared to the same nucleic acid molecule in a random coil or other non-structured state. In some configurations, the tertiary structure (i.e. the helical twist or direction of the polynucleotide strand) of a nucleic acid nanostructure can be configured to be more dense than the same nucleic acid molecule in a random coil or other non-structured state. Nucleic acid nanostructures may include deoxyribonucleic acid (DNA), ribonucleic acid (RNA), peptide nucleic acid (PNA), other nucleic acid analogs, and combinations thereof. Nucleic acid nanostructures may have naturally-arising or engineered secondary, tertiary, or quaternary structures. A structured nucleic acid particle can contain at least one of: i) a moiety that is configured to couple an analyte to the nucleic acid nanostructure, ii) a moiety that is configured to couple the nucleic acid nanostructure to another object such as another SNAP, a solid support or a surface thereof, iii) a moiety that is configured to provide a chemical or physical property or characteristic to a nucleic acid nanostructure, or iv) a combination thereof. Exemplary SNAPs may include nucleic acid nanoballs (e.g., DNA nanoballs), nucleic acid nanotubes (e.g., DNA nanotubes), and nucleic acid origami (e.g., DNA origami). A SNAP may be functionalized to include one or more reactive handles or other moieties. A SNAP may comprise one or more incorporated residues that contain reactive handles or other moieties (e.g., modified nucleotides).

In some embodiments set forth herein, the term “nucleic acid origami” may refer to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

In some embodiments set forth herein, the term “nucleic acid tag” may refer to a nucleic acid molecule or sequence that is encoded with information that identifies or characterizes an object with which it is associated. A nucleic acid tag can be associated with an object via a connection. The connection can be physical, including for example, attachment, colocalization, diffusional contact or the like. Non-physical connections can include, for example, knowledge of a past interaction, knowledge of a shared characteristic, knowledge of common manipulations, knowledge of origin or the like. The nucleic acid tag can be, for example, DNA, RNA or analogs thereof. The length of the tag sequence can be at least about 5, 8, 10, 15, 20, 25, 30, 40, 50, 75, 100 or more nucleotides. Alternatively or additionally, the length of the tag sequence can be at most about 100, 75, 50, 40, 30, 25, 20, 15, 10, 8, 5 or fewer nucleotides.

In some embodiments set forth herein, the term “paratope” may refer to a molecule or part of an affinity reagent, which recognizes or binds specifically to an epitope. A paratope may include an antigen binding site of an antibody. A paratope may include at least 1, 2, 3, or more complementarity-determining regions of an antibody. A paratope need not necessarily be present in nor derived from an antibody, for example, instead being present in a nucleic acid aptamer, lectin, streptavidin, miniprotein or other affinity reagent. A paratope need not necessarily participate in, nor be capable of, eliciting an immune response.

In some embodiments set forth herein, the terms “protein” and “polypeptide” may refer synonymously to a molecule comprising two or more amino acids joined by a peptide bond. A protein may also be referred to as a polypeptide, oligopeptide or peptide. Although the terms “protein,” “polypeptide,” “oligopeptide” and “peptide” may optionally be used to refer to molecules having different characteristics, such as amino acid composition, amino acid sequence, amino acid length, molecular weight, origin of the molecule or the like, the terms are not intended to inherently include such distinctions in all contexts. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

In some embodiments set forth herein, the term “single,” when used in reference to an object such as an analyte, may mean that the object is individually manipulated or distinguished from other objects. A single analyte can be a single molecule (e.g., single protein), a single complex of two or more molecules (e.g., a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

In some embodiments set forth herein, the term “site” may refer to a location in an array where a particular analyte (e.g., protein, peptide or unique identifier label) is present. An address can contain a single analyte or, alternatively, it can contain a population of several analytes. Optionally, a population of analytes at an address can be the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

In some embodiments set forth herein, the term “solid support” may refer to a substrate that is insoluble in aqueous liquid. Optionally, the substrate can be rigid. The substrate can be non-porous or porous. The substrate can optionally be capable of taking up a liquid (e.g., due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor®, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

In some embodiments set forth herein, the term “tag” may refer to a molecule or moiety having a recognizable structure that is attached to a macromolecule. A tag may comprise a detectable or transferrable information that facilitates spatial detection of locations containing the macromolecule to which the tag is attached. A tag may comprise an epitope or recognizable sequence of residues. A tag may be bound by an affinity agent. A tag may include a peptide tag or a nucleic acid tag.

In some embodiments set forth herein, the term “tether” may refer to a molecule or moiety that is configured to interact with a docker or that is interacting with a docker. A tether can be a moiety of a substance, object, molecule, solid support, address, particle, or bead. A tether can include a polymer, nucleic acid strand, nucleic acid duplex, nucleotide sequence, protein, affinity reagent, epitope, paratope, receptor, ligand or the like. A tether can interact with a docker via covalent or non-covalent bonding.

The embodiments set forth below and recited in the claims can be understood in view of the above definitions.

### Macromolecular Display Systems and Methods

Provided herein are systems for displaying macromolecules on solid supports that facilitate contact between structures, moieties, or epitopes of the macromolecules and a fluidic medium surrounding the macromolecules. Also provided herein are systems for displaying macromolecules on solid supports that facilitate manipulation of macromolecular conformations or morphologies. In some cases, the macromolecules are attached to particles that facilitate attachment of the macromolecules to a solid support and control the conformations or morphologies of the macromolecules.

The systems set forth herein may be useful for the display of any type of macromolecule. The systems set forth herein may be especially useful for the display of polymeric macromolecules, and in particular polymeric macromolecules having linear chains, although the systems may also be useful for displaying branched or dendrimeric polymeric macromolecules. Polymeric macromolecules may include any concatenated, covalently bonded group of monomers or residues. Polymeric macromolecules can include biomolecules, such as polypeptides, nucleic acids, and polysaccharides, as well as synthetic, engineered, or naturally occurring non-biological polymeric macromolecules. Some embodiments set forth herein may be exemplified by biological macromolecules, but it shall be understood that other macromolecules may be readily substituted into described systems and methods. In some cases, macromolecules may be analytes, for example analytes obtained from a sample. It may be advantageous to assay analytes in an array format (e.g., a single-analyte array).

A macromolecule can include at least one moiety or functional group that facilitates attachment of the macromolecule to a solid support or a particle that is configured to couple to a solid support. Preferably, a macromolecule can include two or more moieties or functional groups that facilitate attachment of the macromolecule to a solid support or a particle that is configured to couple to a solid support. A moiety or functional group that facilitates attachment of a macromolecule to a solid support may be a terminal moiety, functional group, residue, or monomer. For example, an N-terminal or C-terminal amino acid of a polypeptide, or a 5′-terminal or 3′-terminal nucleotide of a nucleic acid may be utilized for attachment of the polypeptide or nucleic acid, respectively, to a solid support or a particle configured to be attached to a solid support. A moiety or functional group that facilitates attachment of a macromolecule to a solid support may be a non-terminal moiety, functional group, residue, or monomer. For example, particular amino acids of a polypeptide molecule contain sidechains comprising a reactive functional group (e.g., arginine, lysine, aspartic acid, glutamic acid, asparagine, glutamine, cysteine, etc.) that may provide useful attachment moieties for facilitating attachment of a polypeptide to a solid support or a particle that is configured to couple to a solid support.

Attachment moieties may be provided to a macromolecule before the macromolecule is attached to a solid support or a particle that is configured to be coupled to a solid support. A macromolecule may be modified (e.g., chemically modified, enzymatically modified) to provide a moiety or functional group that facilitates attachment of the macromolecules to a solid support or a particle that is configured to couple to a solid support. In some cases, a moiety or functional group of a macromolecule may be modified (e.g., chemically modified, enzymatically modified) to form an altered moiety or functional group that provides a more useful attachment chemistry. For example, certain amino acid sidechains can be modified to form Click-type reactive functional groups that facilitate attachment of other moieties to the polypeptide via Click-type reactions. In some cases, two or more moieties or functional groups of a macromolecule may be modified (e.g., chemically modified, enzymatically modified) to form altered moieties or functional groups that provides a more useful attachment chemistries. In some cases, two or more moieties or functional groups of a macromolecule may be modified in the same fashion (i.e., producing the same moiety or functional group). In other cases, two or more moieties or functional groups of a macromolecule may be modified in the same fashion. For example, it may be useful to provide a first attachment site with a first functional group that differs from a second attachment site with a second functional group, in which the first functional group and the second functional group have orthogonal reactive chemistries (e.g., an amine and a carboxylate). The modification of macromolecules to provide useful attachment moieties or functional groups is described in U.S. Pat. No. 11,203,612 which is herein incorporated by reference in its entirety.

Additional moieties may be attached to a macromolecule to facilitate attachment of the macromolecule to a solid support or a particle that is configured to be coupled to a solid support. A moiety attached to a macromolecule may comprise a tag (e.g., a peptide tag, a nucleic acid tag, etc.). A tag may comprise a residue sequence (e.g., an amino acid sequence, a nucleotide sequence) that can be recognized and/or bound by a binding entity that recognizes and/or binds to the residue sequence. A moiety attached to a macromolecule may comprise a component of a receptor-ligand binding pair (e.g., streptavidin/biotin, SpyCatcher-SpyTag, SnoopCatcher-SnoopTag, SdyCatcher-SdyTag, etc.). A moiety attached to a macromolecule may comprise a nucleic acid (e.g., a single-stranded nucleic acid, a double-stranded nucleic acid, a combination thereof). A moiety attached to a macromolecule may comprise a linking moiety or a spacing moiety (e.g., a polyethylene glycol moiety, an alkyl moiety, a nucleic acid, a peptide, etc.). A linking or spacing moiety may provide a separation gap (e.g., at least about 5 nanometers (nm), 10 nm, 20 nm, 30 nm, 50 nm, 100 nm, etc.) between the macromolecule and a moiety or functional group that facilitates attachment of the macromolecule to a solid support or a particle that is configured to be coupled to a solid support.

A macromolecule may be attached to a site of a solid support. Preferably, the site is a site of a plurality of sites of an array, in which individual sites of the plurality of sites are each optically resolvable from each other site. A macromolecule may be attached to a site having one or more attachment sites that are configured to covalently or non-covalently attach the macromolecule to the solid support. Preferably, a site will have two or more attachment sites (e.g., at least 2, 3, 4, 5, 10, 20, 50, 100, or more than 100 attachment sites) that are configured to covalently or non-covalently attach the macromolecule to the solid support.

It may be advantageous to provide a particle that couples to a site, in which the particle further comprises at least one attachment site that is configured to attach to a macromolecule. A macromolecule may be attached to a particle having one or more attachment sites that are configured to covalently or non-covalently attach the macromolecule to the solid support. Preferably, a particle will have two or more attachment sites (e.g., at least 2, 3, 4, 5, 10, 20, 50, 100, or more than 100 attachment sites) that are configured to covalently or non-covalently attach the macromolecule to the particle. The particle can further comprise one or more moieties that couple the particle to a site of a solid support. Nucleic acid particles may be useful for displaying macromolecules due to the tunable architectures that can be achieved. Attachment sites can be provided on nucleic acid particles at positions with known or designed separation distances between the attachment sites. A nucleic acid particle can be provided with one or more attachment sites that are oriented in a different direction than moieties that couple the nucleic acid particle to a site of a solid support. For example, a nucleic acid particle can be provided with a first face and a second face, in which the first face and the second face are substantially parallel and opposed, and in which moieties attached to the first face are oriented in a substantially opposite direction to moieties attached to the second face. Useful nucleic acid particles for coupling macromolecules to solid supports are described in U.S. Pat. Nos. 11,203,612, and 11,505,796, each of which is herein incorporated by reference in its entirety.

A macromolecule may be attached to a particle (e.g., a nucleic acid particle) before the particle is coupled to a site on a solid support. Alternatively a macromolecule may be attached to a particle (e.g., a nucleic acid particle) after the particle is coupled to a site on a solid support. In some cases, a first attachment moiety or functional group of a macromolecule may be attached to a particle before the particle is coupled to a site of a solid support, then a second attachment moiety or functional group of the macromolecule may be attached to the particle after the particle is coupled to the site of the solid support.

The macromolecule may be attached to a site of a solid support or a particle by forming a covalent or non-covalent interaction between an attachment moiety or functional group of the macromolecule and a complementary attachment moiety or functional group at an attachment site of the site of the solid support or the particle. In some cases, a macromolecule may be attached to a site of a solid support or a particle by forming two or more covalent or non-covalent interactions between pairs of attachment moieties or functional groups of the macromolecule and complementary attachment moieties or functional groups at an attachment site of the site of the solid support or the particle.

An attachment site provided at a site may be configured to provide a separation gap between a solid support comprising the site and a macromolecule attached to the attachment site. An attachment site may comprise a separating moiety that provides the separation gap between the solid support comprising the site and the macromolecule attached to the attachment site. A separating moiety may comprise a rigid linker or a flexible linker. A rigid linker may comprise a solid material that is formed from or disposed on a solid support (e.g., etched posts or pillars). A rigid linker may comprise a polymeric molecule, such as a double-stranded nucleic acid or a non-saturated alkyl moiety. A flexible linker may comprise a polymeric molecule, such as PEG, single-stranded nucleic acids, peptides, or saturated alkyl moieties. A separating moiety may provide a separation gap (e.g., at least about 5 nanometers (nm), 10 nm, 20 nm, 30 nm, 50 nm, 100 nm, etc.) between the macromolecule and a surface of the solid support containing the site comprising the separating moiety.

In a useful configuration, a rigid linker may be formed by hybridizing a first nucleic acid attachment moiety to a second complementary nucleic acid attachment moiety. The double-stranded nucleic acid formed by the hybridization of the first nucleic acid strand to the second nucleic acid strand may form a rigid spacing moiety between a site or a particle attached thereto and a macromolecule. Alternatively, a rigid linker or a flexible linker may be formed by joining a first nucleic acid attachment moiety at a first attachment site to a second nucleic acid attachment moiety of a macromolecule utilizing a nucleic acid joining strand. The nucleic acid joining strand can comprise a first nucleotide sequence that hybridizes to the first nucleic acid attachment moiety and a second nucleotide sequence that hybridizes to the second nucleic acid attachment moiety. If the joining strand or the attachment moieties further comprise portions of single-stranded nucleic acids after the hybridizing, the formed linker may exhibit rigid and/or flexible characteristics depending upon the length of the single-stranded portions.

Systems for macromolecule display provided herein may be advantageous for manipulating the conformation or morphology of the macromolecule when the macromolecule is contacted with a fluid medium. In particular, a system may display a macromolecule in a conformation that facilitates contact between a fluid medium and a residue, monomer, functional group, or a plurality thereof that may be sequestered away from the fluid medium in a naturally-occurring conformation or morphology of the macromolecule. For example, a system provided herein may display a polypeptide such that an epitope that is typically sequestered in an internal portion of the polypeptide molecule (e.g., an epitope containing one or more hydrophobic amino acid sidechains) in a particular fluid medium will be accessible to the fluid medium and to reagents in the fluid medium. Alternatively or additionally, a system may display a macromolecule in a conformation or morphology that does not change substantially when a first fluid medium contacted to the macromolecule is exchanged for a second fluid medium. For example, a protein displayed on a system provided herein may be held in an extended conformation that inhibits folding or re-folding of the protein regardless of the fluid medium contacted to the protein.

It is readily recognized that the characteristic dimension (e.g., length, width, height, diameter) of a macromolecule, such as a polymeric chain, can be substantially less than the extended length of the macromolecule. Returning to FIGS. 1A-1C, it can be seen that a macromolecule may exist within a range of characteristic dimensions between its contour length, L, and a characteristic dimension for its most compact state. The conformation and associated characteristic dimensions of a macromolecule can be associated to a fluid medium containing the macromolecule. For many macromolecules, the extended length is readily predictable based upon the number of monomers or residues present in the macromolecules. For example, in an aqueous medium, proteins can have a contour length of about 3.5 Angstroms (Å) per amino acid, B-form deoxyribonucleic acids can have a contour length of about 3.4 Å per nucleotide, polyethylene glycol molecules can have a contour length of about 2.8 Å per monomer, and polyethylene molecules can have a contour length of about 1.5 Å per monomer.

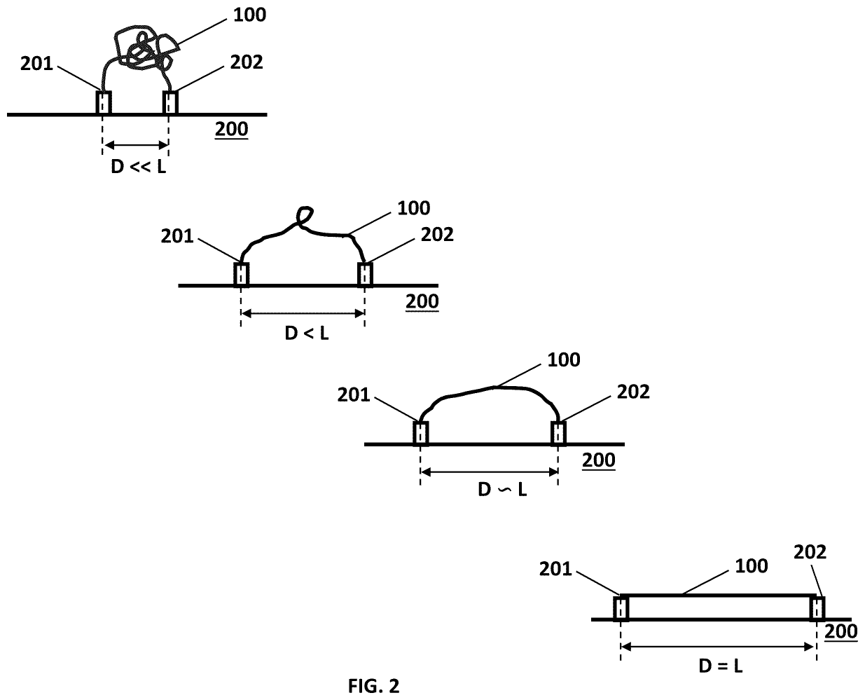

Accordingly, it may be advantageous to display a macromolecule on a solid support in a conformation that extends the macromolecule or a portion thereof to a characteristic dimension close to the contour length for the macromolecule or the portion thereof, wherein the characteristic dimension is longer than the longest dimension of the macromolecule in its native or folded state. FIG. 2 depicts examples of a polymeric chain 100 of contour length L that is secured to two attachment sites 201 and 202 that are separated by a distance D on a surface 200 (e.g., a surface of a solid support, a surface of a particle). In the upper leftmost configuration (D<<L), the smaller distance between attachment sites 201 and 202 allows formation of a folded structure that may sequester certain monomers, residues, or moieties of the polymeric chain 100. In the second configuration (D<L), the attachment sites 201 and 202 are located at the largest separation at which some folding of the polymeric chain 100 can occur. In the third configuration (D˜L), the polymeric chain 100 is sufficiently extended between the attachment sites 201 and 202 to inhibit folding of the polymeric chain 100, but polymeric chain is not fully extended. In the lowermost right configuration (D=L), the polymeric chain 100 is fully extended to its full contour length between the attachment sites 201 and 202.

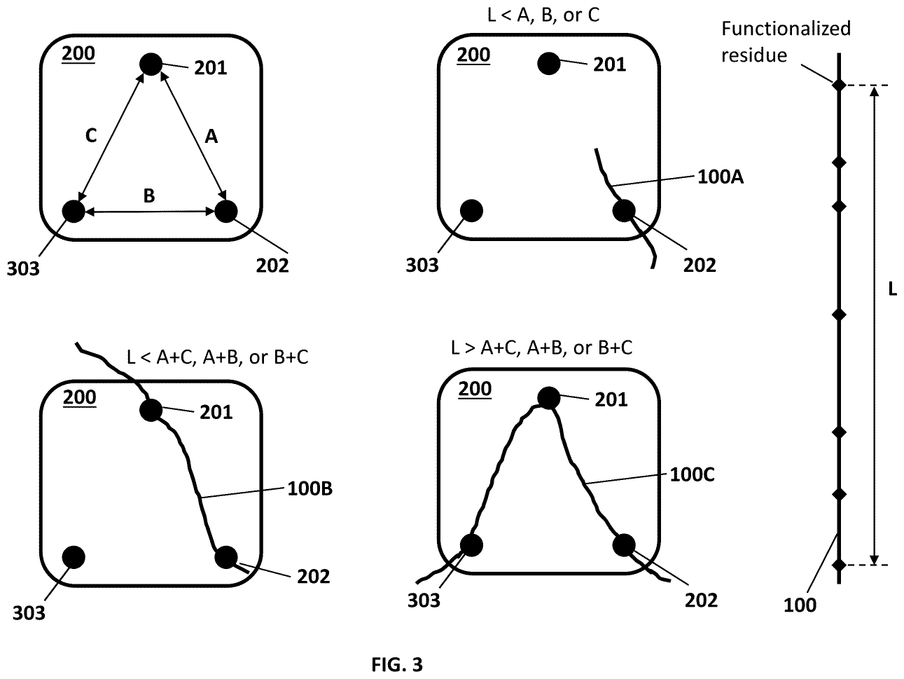

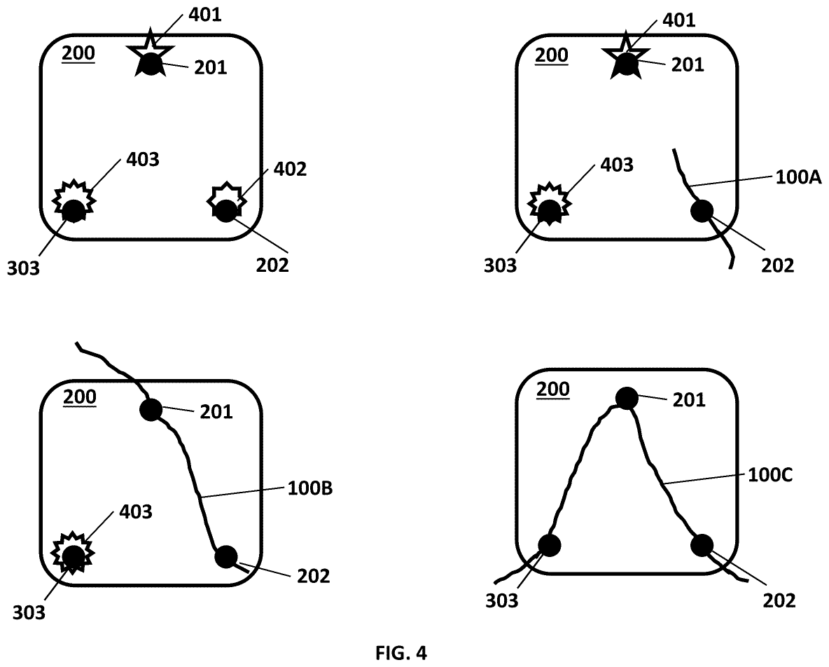

For a plurality of macromolecules with minimal dispersity of size (e.g., polymeric chain length), an ideal separation distance for two attachment sites can be predicted to secure the macromolecules in a nearly fully-extended or fully-extended conformation (per FIG. 2, D˜L or D=L). In more complex samples, macromolecules may have a dispersity of sizes or lengths. In such cases, it may be preferable to provide additional attachment sites that can facilitate attachment of macromolecules of differing sizes in their respective extended conformations. FIG. 3 illustrates a surface 200 (e.g., a surface of a solid support, a surface of a particle) like that depicted in FIG. 2, but further comprising a third attachment site 303. The first attachment site 201 and the second attachment site 202 are separated from the third attachment site 303 by linear distances C and B, respectively. The first attachment site 201 is separated from the second attachment site 202 by a linear distance A. On the far right of FIG. 3, a polymeric chain 100 is depicted, with functionalized monomers, residues, or moieties that can be utilized for attachment of the polymeric chain 100 to an attachment site of the surface 200. The polymeric chain 100 can have an effective contour length L that represents the distance between the furthest separated functionalized monomers, residues, or moieties. If both of the termini of the polymeric chain 100 are utilized for attachment, then L would be the contour length of the full macromolecule. In the upper right most surface configuration, a short polymeric chain 100A is attached to the second attachment site 202. Because L<A, B or C, the polymeric chain 100A cannot be attached to two attachment sites simultaneously. In the lower leftmost configuration, the polymeric chain 100B is long enough to attach to two attachment sites simultaneously (i.e., L>A or B or C) but is not long enough to attach to three attachment sites simultaneously (i.e., L<A+B or A+C or B+C). In the lower rightmost configuration, the polymeric chain 100C is long enough to attach to three attachment sites simultaneously (i.e., L>A+B or A+C or B+C).

For a system of macromolecular display set forth herein, the size of a macromolecule may be determinable or estimable by a quantity of attachments sites to which the macromolecule is attached. FIG. 4 depicts the configurations of FIG. 3 with detectable labels attached to unoccupied attachment sites. In the upper leftmost configuration, attachment sites 201, 202, and 203 comprise detectable labels 401, 402, and 403, respectively. If the attachment sites have orthogonal attachment chemistries, detectable labels 401, 402, and 403 may differ (e.g., with respect to detectable signal). Alternatively, if the attachment sites have the same attachment chemistries, a quantity of unoccupied attachment sites may be estimated by step change in signal from the detectable labels. The quantity of detected signals (or the magnitude of detected signal) will decrease as more attachment sites are occupied by an attached polymeric chain 100. In the lower rightmost configuration, no signal from detectable labels would be detected because all attachment sites are occupied by the longest polymeric chain 100C.

In some cases, a sensing group may be bound to an unoccupied attachment site. A sensing group may comprise a complementary attachment moiety and a detectable label. The complementary attachment moiety may couple with an attachment moiety at an attachment site. Presence of a bound sensing group at a site or a particle attached thereto may be determined by detection of a signal (e.g., a fluorescent signal) from a detectable label of the sensing group. Absence of a bound sensing group at a site or a particle attached thereto may be determined by absence of a signal (e.g., a fluorescent signal) from a detectable label of the sensing group.

The present disclosure provides for array containing sites for displaying macromolecules, in which individual sites of the array each comprise two or more attachment sites, and in which a single macromolecule is attached to at least two of the two or more attachment sites. The two or more attachment sites may be formed on a solid support at an address corresponding to an array site. Preferably, one or more particles (e.g., nucleic acid nanoparticles) may be provided at a site, in which each particle contains at least one attachment site (e.g., at least 2, 3, 4, 5, 6, 7, 8, 9, 10, or more than 10 attachment sites). Most preferably, one and only one particle will be coupled to a single array site, in which the particle comprises two or more attachment sites. Alternatively, two or more particles can be coupled to a single array site, in which individual particles of the two or more particles each provide at least one attachment site.

For an individual site or a particle provided at a site, two attachment sites may be separated by a distance. Two attachment sites at an individual site or particle may be separated by a distance of at least about 5 nanometers (nm), 10 nm, 15 nm, 20 nm, 25 nm, 30 nm, 40 nm, 50 nm, 60 nm, 70 nm, 80 nm, 90 nm, 100 nm, 120 nm, 140 nm, 160 nm, 180 nm, 200 nm, 250 nm, 300 nm, 400 nm, 500 nm, or more than 500 nm. Alternatively or additionally, two attachment sites at an individual site or particle may be separated by a distance of no more than about 500 nm, 400 nm, 300 nm, 250 nm, 200 nm, 180 nm, 160 nm, 140 nm, 120 nm, 100 nm, 90 nm, 80 nm, 70 nm, 60 nm, 50 nm, 40 nm, 30 nm, 25 nm, 20 nm, 15 nm, 10 nm, 5 nm, or less than 5 nm. The separation distance between two attachment sites at an individual site or a particle coupled thereto may be proportional to the characteristic dimension (e.g., length, width, diameter) of the attachment site or particle, such as at least about 5%, 10%, 15%, 20%, 25%, 30%, 35%, 40%, 45%, 50%, 55%, 60%, 65%, 70%, 75%, 80%, 85%, 90%, 95%, or more than 95% of the characteristic dimension of the attachment site or particle. Alternatively or additionally, the separation distance between two attachment sites at an individual site or a particle coupled thereto may be no more than about 95%, 90%, 85%, 80%, 75%, 70%, 65%, 60%, 55%, 50%, 45%, 40%, 35%, 30%, 25%, 20%, 15%, 10%, 5%, or less than 5% of the characteristic dimension of the attachment site or particle. For sites or particles that are asymmetric (i.e., having a non-equal length and width or a non-uniform diameter), the separation distance between two attachment sites may be characterized with respect to the maximum or minimum characteristic dimension. For a plurality of sites or particle coupled to sites, an average separation distance may be characterized in absolute or proportional terms as the average separation distance of all sites or particles of the plurality of sites or particles.

A particle may be especially useful for the systems and methods disclosed herein if it comprises two or more attachment sites in known or fixed locations. Further, a particle may be useful if it contains at least two faces, in which each of the two faces individually provides a differing spatial orientation of moieties attached to the face. For example, it may be preferable for a particle to have first face containing at least one surface-coupling moiety and a second face that is substantially opposed to the first face containing attachment sites that are configured to couple a macromolecule. Accordingly, when such a particle is coupled by the surface-coupling moiety of the first face to a site or solid support, the second face, and optionally a macromolecule attached to the second face, is oriented in a direction opposed or outward from the site or solid support. Nucleic acid particles may be especially useful particles for coupling macromolecules to array sites. Nucleic acid particles can be designed with tunable architectures that facilitate location of structural features such as attachment sites and spacing moieties. Further, nucleic acid nanoparticles, such as nucleic acid origami, can be designed to provide multiple faces that provide control of the orientations of moieties attached to the nucleic acid particle. Useful nucleic acid particles are described in U.S. Pat. No. 11,505,796, which is incorporated by reference in its entirety.

A face of a particle may have a characteristic dimension (e.g., length, width, diameter) of at least about 10 nm, 15 nm, 20 nm, 25 nm, 30 nm, 40 nm, 50 nm, 60 nm, 70 nm, 80 nm, 90 nm, 100 nm, 120 nm, 140 nm, 160 nm, 180 nm, 200 nm, 250 nm, 300 nm, 400 nm, 500 nm, or more than 500 nm. Alternatively or additionally, a face of a particle may have a characteristic dimension of no more than about 500 nm, 400 nm, 300 nm, 250 nm, 200 nm, 180 nm, 160 nm, 140 nm, 120 nm, 100 nm, 90 nm, 80 nm, 70 nm, 60 nm, 50 nm, 40 nm, 30 nm, 25 nm, 20 nm, 15 nm, 10 nm, or less than 10 nm. The characteristic dimension of a face of a particle that is coupled to a site may be smaller, larger, or substantially equal to the characteristic dimension of the site. A characteristic dimension of a first face of a particle may be smaller, larger, or substantially equal to a characteristic dimension of a second face of the particle.

In an aspect, provided herein is a composition, comprising: a) a particle comprising a first face and a second face, in which the first face is substantially opposed to the second face, in which the second face comprises a first attachment site containing a first attachment moiety, and in which the second face further comprises a second attachment site containing a second attachment moiety, b) a plurality of coupling moieties coupled to the first face, and c) a macromolecule, in which the macromolecule comprises a first complementary attachment moiety and a second complementary attachment moiety, in which the first complementary attachment moiety is attached to the first attachment moiety, and in which the second complementary attachment moiety is attached to the second attachment moiety.

In another aspect, provided herein is a method, comprising: a) contacting a macromolecule to a particle, in which the particle comprises a first attachment site comprising a first attachment moiety, and a second attachment site comprising a second attachment moiety, b) attaching a first complementary attachment moiety of the macromolecule to the first attachment moiety of the particle, and attaching a second complementary attachment moiety of the macromolecule to the second attachment moiety of the particle, and c) coupling the particle to a site of a solid support.

In some cases, contacting the macromolecule to the particle can occur before coupling the particle to the solid support. For example, a macromolecule may be contacted to and coupled to a particle before the particle is delivered to a site. In such cases, the particle and macromolecule can both be in fluid phase when contacted with each other. Alternatively, contacting a macromolecule to a particle can occur after coupling the particle to the solid support. In such cases, the particle is immobilized and the macromolecule can be provided in fluid phase. Optionally, an array of particles may be formed, in which individual sites of the array each contain one and only one particle. Thereafter, a single macromolecule may be attached to each individual particle at a respective individual site.

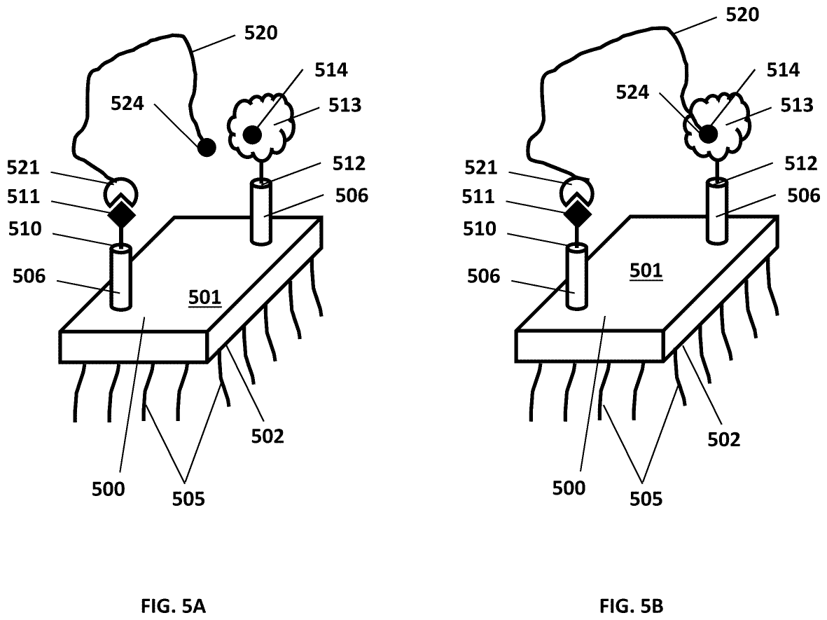

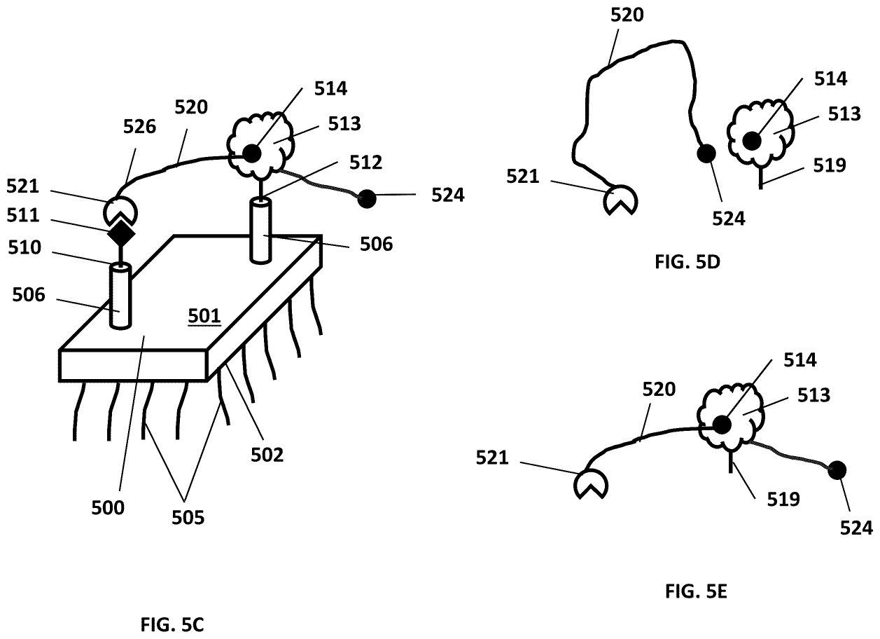

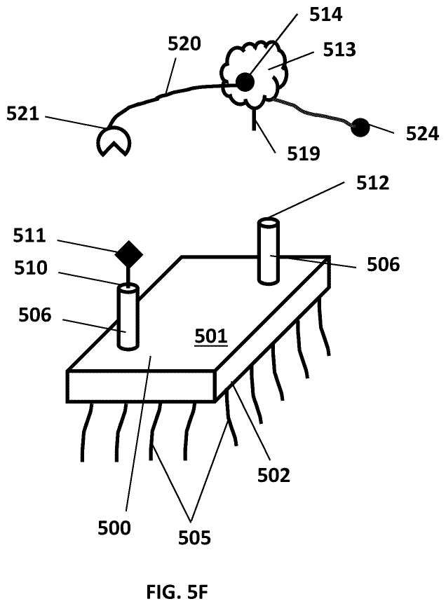

FIGS. 5A-5C depict a method of forming a particle comprising a coupled macromolecule. FIG. 5A shows a particle 500 (e.g., a nucleic acid particle) having a first face 501 (oriented upward as depicted) and a second face 502 (oriented downward and not shown as depicted). The first face comprises a first attachment site 510 that is attached to the particle 500 by an optional spacing moiety 506 and a second attachment site 512 that is attached to the particle 500 by an optional spacing moiety 506. The first attachment site 510 comprises a first attachment moiety 511 that is configured to covalently or non-covalently attach a macromolecule 520 to the particle 500. The second attachment site 512 comprises a second attachment moiety 513 that is configured to non-covalently attach a macromolecule 520 to the particle 500. In the configuration shown in FIG. 5A, the second attachment moiety 513 may comprise an analyte-binding group (e.g., an analyte-binding polypeptide, an analyte-binding nucleic acid) containing an analyte-binding site 514. A macromolecule 520 is partially coupled to the particle 500 by attaching a first complementary attachment moiety 521 to the first attachment moiety 511 of the first attachment site 510. The macromolecule further comprises a second complementary attachment moiety 524 that is not coupled. FIG. 5B depicts a second configuration, in which the second complementary attachment moiety 524 of the macromolecule 520 has coupled to the analyte-binding site 514 of the second attachment moiety. FIG. 5C depicts a third optional configuration, in which the macromolecule 520 has been extended by passage of the polymeric chain of the macromolecule 520 through the second attachment moiety 513. The portion 526 of the macromolecule 520 coupled between the first attachment site 510 and the second attachment site 512 may have a length that approaches the contour length of the portion 526 of the macromolecule 520 as more of the macromolecule 520 is passed through the second attachment moiety 513.

FIGS. 5D-5F depict an alternative method of forming a particle comprising a coupled macromolecule. FIG. 5D shows a macromolecule 520 contacted with a second attachment moiety 513 (e.g., an analyte-binding group) comprising an analyte-binding site 514 and a coupling moiety 519 that is configured to bind to a second attachment site 512. FIG. 5E depicts a second configuration in which the second complementary coupling moiety 524 of the macromolecule 520 has coupled to the analyte-binding site 514 and passed through the second attachment moiety 513. FIG. 5F depicts a third configuration, in which the complex comprising the macromolecule 520 and the second attachment moiety 513 is contacted to a particle as described in FIG. 5A. Coupling of the first complementary attachment moiety 521 to the first attachment moiety 511 and coupling of the coupling moiety 519 to the second attachment site 512 will provide the configuration depicted in FIG. 5C.

An attachment moiety can comprise an analyte-binding group. An analyte-binding group may comprise a binding entity that specifically or non-specifically couples to a macromolecule or a moiety attached thereto. For example, an analyte-binding group may specifically bind to an epitope of a macromolecule. Alternatively, an analyte-binding group may be capable of binding to many epitopes of macromolecules. In some cases, an analyte-binding group may bind to a tag (e.g., a nucleic acid tag, a peptide tag) or other moiety (e.g., a binding ligand or substrate) attached to a macromolecule. Accordingly, a useful analyte-binding group may comprise a protein. For polypeptide macromolecules, an analyte-binding group may comprise a polypeptide-binding protein. Unfoldases (e.g., AAA+unfoldase, CLpX unfoldase, CDC48 unfoldase, etc.) may be especially useful for both binding to a polypeptide molecule and extending the polypeptide molecule by disrupting secondary and/or tertiary structures. Other useful analyte-binding proteins that bind to polypeptides can include enzymes (e.g., proteases, methylases, phosphorylases, glycases, etc.) that optionally have been engineered to deactivate the active site. For nucleic acids, an analyte-binding group may comprise a nucleic acid-binding protein. For example, helicases, transcription factors, and histones may be useful for binding to nucleic acid strands. Other useful analyte-binding proteins that bind to polypeptides can include enzymes (e.g., polymerases, reverse transcriptases, ligases, etc.) that optionally have been engineered to deactivate the active site.

In some cases, it may be useful to attach a tag (e.g., a nucleic acid tag, a peptide tag) to a macromolecule to facilitate binding to a macromolecule. A tag may comprise a sequence of residues (e.g., a nucleotide sequence, an amino acid sequence) that is bound by an analyte-binding group. For example, certain nucleotide sequences are bound by transcription factors. Likewise, certain amino acids sequences may be preferentially bound by unfoldases.

In some cases, after coupling an analyte-binding group to a macromolecule, a third complementary attachment moiety of the macromolecule may be attached to an attachment moiety at the attachment site containing the analyte-binding group. For example, the analyte-binding group may extend a macromolecule, thereby bringing a complementary attachment moiety in sufficient proximity to the attachment site to facilitate covalent or non-covalent coupling of the macromolecule to the attachment site. Alternatively, after coupling an analyte-binding group to a macromolecule, a third complementary attachment moiety of the macromolecule may be attached to an attachment moiety at an attachment site that does not contain the analyte-binding group. For example, the analyte-binding group may be coupled to a macromolecule at an internal epitope, tag, or residue, and the macromolecule may further comprise a pendant portion that contains a complementary attachment moiety that can become bound to an attachment moiety at an unoccupied attachment site.

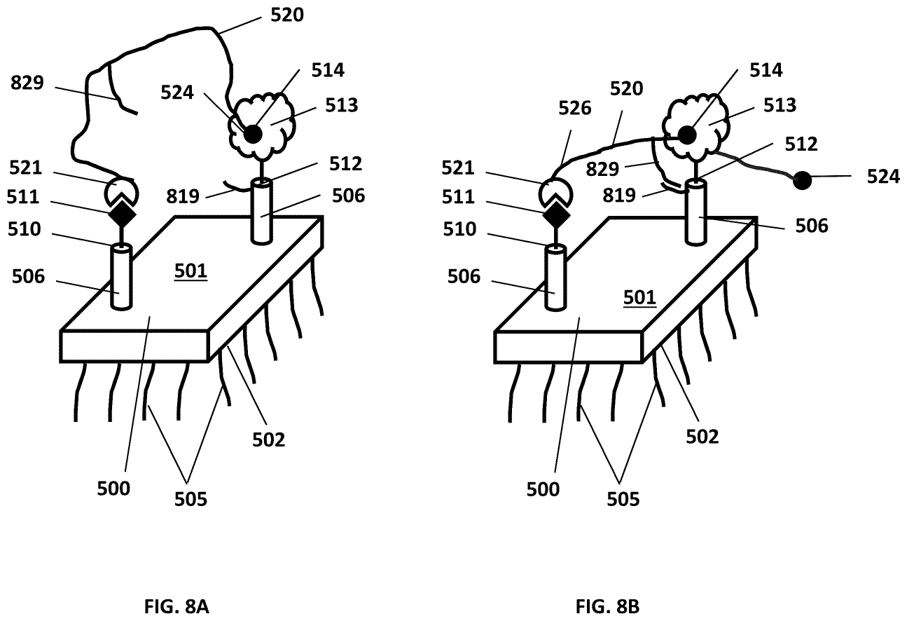

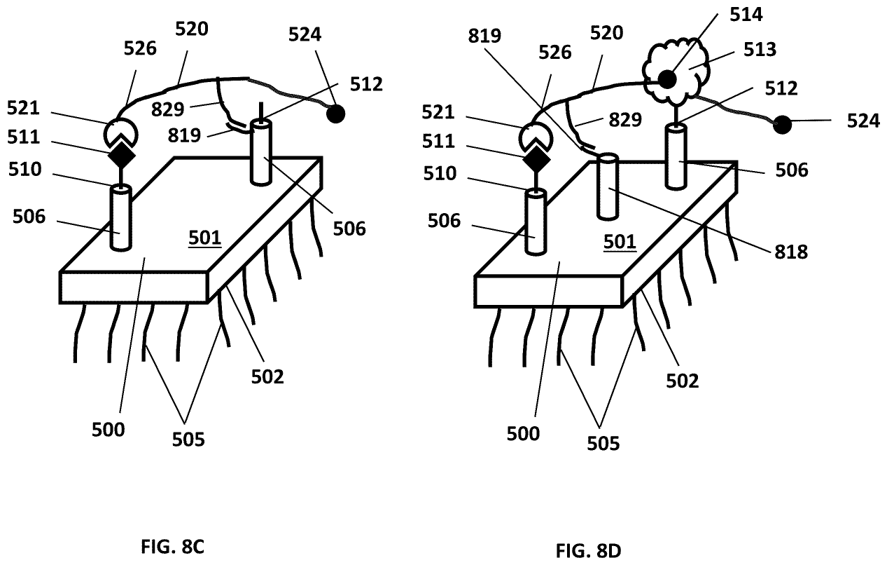

FIGS. 8A-8D illustrate aspects of attaching a macromolecule to an attachment site when the macromolecule is coupled to an analyte-binding group. FIG. 8A depicts a configuration of a system similar to the system described for FIG. 5A. The macromolecule 520 may further comprise a complementary third attachment moiety 829 that is configured to form a binding interaction with a third attachment moiety 819. In the depicted configuration, the complementary third attachment moiety 829 may not form a binding interaction with the third attachment moiety 819 due to a distance between the two moieties or due to conformational hindrance of the macromolecule 520. FIG. 8B depicts a second configuration in which the macromolecule 520 has been extended by the analyte-binding group 513. In the second configuration, the third complementary attachment moiety 829 is sufficiently close to the third attachment moiety 819 to form a binding interaction between the two moieties. FIG. 8C depicts a third configuration, in which the analyte-binding group 513 has been released from the second attachment site 512. The macromolecule 520 remains attached to the first attachment site 510 and the second attachment site 512 in part due to the binding interaction between the third complementary attachment moiety 829 and the third attachment moiety 819. FIG. 8D depicts an alternative configuration in which the third complementary attachment moiety 829 is attached to the third attachment moiety 819 at a third attachment site 818.

A method set forth herein may comprise a step of detaching an analyte-binding group from an attachment site. An analyte-binding group may be detached after the analyte-binding group has facilitated the alteration of a conformation or morphology of a macromolecule. An analyte-binding group may be attached to an attachment site by a labile moiety (e.g., a reactive moiety, a photolabile moiety, a single- or double-stranded nucleic acid, a peptide moiety, etc.). Accordingly, detaching an analyte-binding group from an attachment site may comprise chemically or enzymatically cleaving a covalent bond of the labile moiety (e.g., contacting a photolabile moiety with light, contacting a labile moiety with a reactive chemical species, contacting a nucleic acid labile moiety with a restriction enzyme, contacting a peptide labile moiety with a protease, etc.). Alternatively, an analyte-binding group may be attached to an attachment site by a non-covalent interaction between an attachment moiety coupled to the attachment site and a complementary attachment moiety coupled to the analyte-binding group (e.g., an oligonucleotide, a component of a ligand-receptor binding pair). Accordingly, detaching an analyte-binding group from an attachment site may comprise dissociating the non-covalent binding interaction between the attachment moiety and the complementary attachment moiety. For example, an oligonucleotide attachment moiety may be de-hybridized from a complementary oligonucleotide attachment moiety attached to an analyte-binding group.

An analyte binding group may be separated from a macromolecule after the analyte-binding group has coupled to the macromolecule. An analyte binding group may be separated from a tag attached to a macromolecule after the analyte-binding group has coupled to the tag. For a protein-based analyte-binding group, separating the analyte-binding group from the macromolecule may comprise denaturing the protein analyte-binding group. Accordingly, a method may comprise a step of contacting a protein with a denaturing or chaotropic species, as set forth herein. Alternatively, certain protein analyte-binding groups may separate from a macromolecule or a tag attached thereto by a terminating sequence of residues. For example, a transcription factor may release from a nucleic acid (e.g., a nucleic acid macromolecule or a nucleic acid tag) if the nucleic acid contains a terminating sequence of nucleotides.

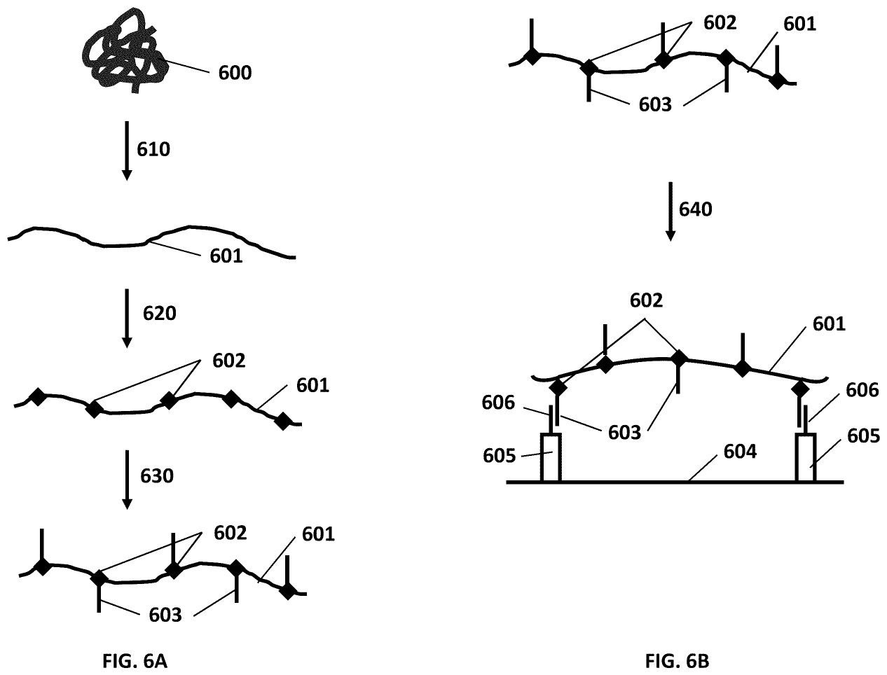

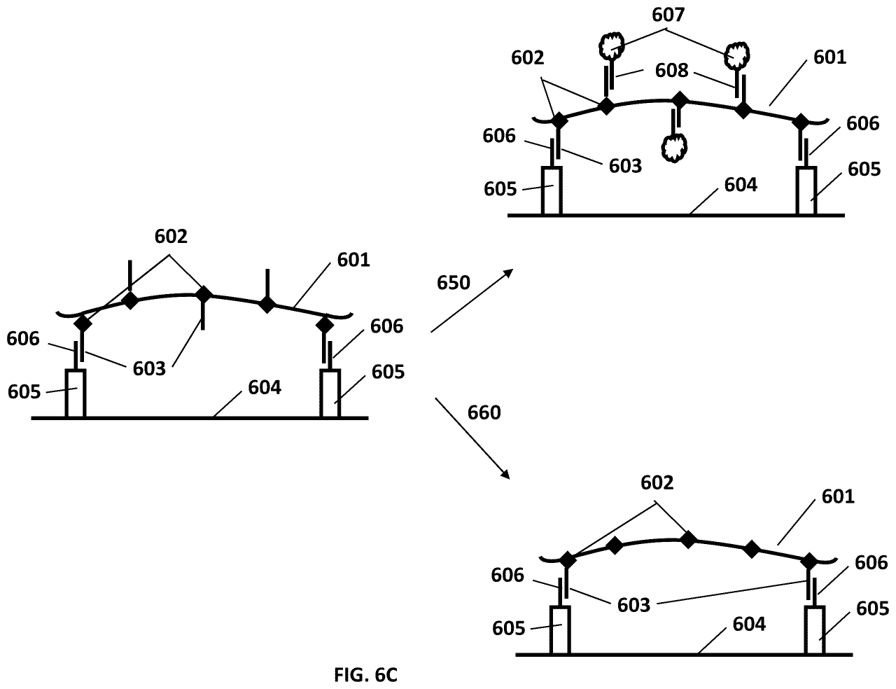

FIGS. 6A-6C depict additional aspects of preparing and/or displaying a macromolecule on a solid support. FIG. 6A depicts a method of preparing a macromolecule for attachment to a solid support. A macromolecule 600 (e.g., a polymeric chain) may be provided in a folded or condensed state. The macromolecule 600 may undergo a denaturation, unfolding, or relaxation process 610 that provides a more extended configuration 601 of the macromolecule 600. The denaturation, unfolding, or relaxation process 610 may occur in the presence of a fluid medium containing a denaturing agent, chaotrope agent, or unfolding enzyme. Alternatively, a denaturation, unfolding, or relaxation process 610 can occur due to a change in a pH or ionic strength of a fluid medium, or by heating of a fluid medium containing the macromolecule 600. Optionally, the extended macromolecule 601 may undergo a chemical modification process 620 that selectively alters certain monomers, residues, or moieties of the extended macromolecule 601 to provide functionalized monomers, residues, or moieties 602. In some cases, a macromolecule 600 or extended macromolecule 601 can intrinsically possess functionalized monomers, residues, or moieties 602. Optionally, the extended macromolecule 601 may undergo attachment 630 of complementary attachment moieties 603 (e.g., oligonucleotide, receptor-ligand pair components, reactive functional groups, etc.), preferably at functionalized monomers, residues, or moieties 602. Optionally, the complementary attachment moieties 603 can be attached to the extended macromolecule 601 by linking moieties or spacing moieties.

FIG. 6B depicts coupling of the extended macromolecule 601 of FIG. 6A to a solid support or a particle attached thereto. The extended macromolecule is contacted 640 to a site or particle 604 containing attachment sites 605. The individual attachment sites each contain an attachment moiety that forms a binding interaction with a complementary attachment moiety 603 of the extended macromolecule 601. FIG. 6C depicts optional steps that may occur after an extended macromolecule 601 has been attached to a site or particle 604. Optionally, occlusion moieties 607 may be attached 650 to unbound complementary attachment moieties 603. The occlusion moieties 607 may comprise polymeric moieties (e.g., PEG molecules) or charged moieties (e.g., cationic polymers, anionic polymers, zwitterionic polymers) that inhibit folding or condensation of the extended macromolecule 601. Individual occlusion moieties 607 may each comprise an attachment moiety 608 that is complementary to the complementary attachment moieties 603 of the extended macromolecule 601. Alternatively, the complementary attachment moieties 603 of the extended macromolecule 601 may undergo a removal process 660 (e.g., chemical digestion, enzymatic digestion, cleavage of a photolabile group of a linking moiety that attaches the complementary attachment moiety to the extended macromolecule 601), thereby providing an extended region of the macromolecule 601 between the attachment sites 605 that is substantially devoid of complementary attachment moieties 603. In some cases, one or more occlusion moieties may be coupled to a macromolecule before attaching the macromolecule to a site or particle.

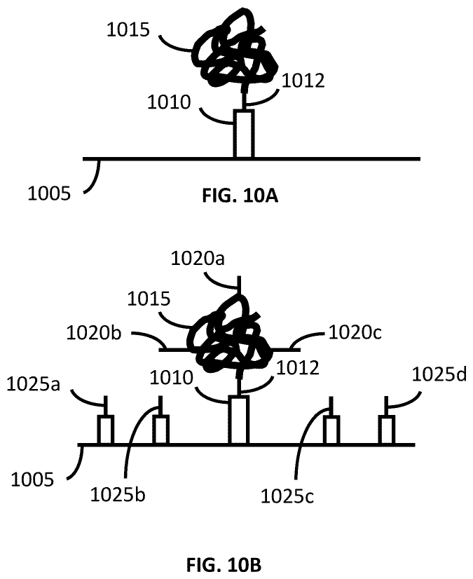

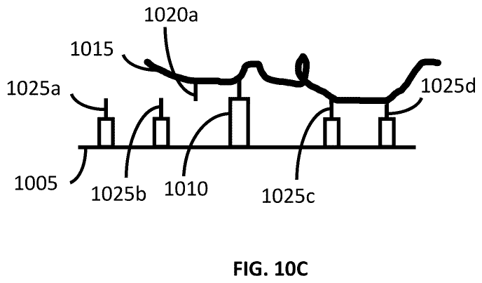

An implementation of some of the previous examples is depicted in FIGS. 10A, 10B, and 10C showing the use of TCO and mTz to retain a macromolecule in a denatured state. In FIG. 10A, a macromolecule 1015 (e.g., a protein) is positioned at an attachment site 1010 of a particle 1005 via an attachment moiety 1012. In FIG. 10A, the macromolecule 1015 is in a folded or globular conformation (or morphology) such that some of the monomers, residues, or moieties may be partially or fully occluded from the surrounding fluidic medium, resulting in affinity reagents unable to access the partially or fully occluded monomers, residues, or moieties.

In FIG. 10B, the macromolecule 1015 and the particle 1005 are subsequently modified with different attachment moieties that can form a binding interaction to attach with each other. For example, the macromolecule 1015 is modified with the addition of attachment moieties 1020a, 1020b, and 1020c, which may be TCO, at different locations around the macromolecule 1015. The particle 1005 is modified with the addition of attachment moieties 1025a, 1025b, 1025c, and 1025d which may be mTz, at different attachment sites. The macromolecule 1015 is then modified to have mTz moieties and the particle 1005 is modified to have TCO moieties. As previously discussed, TCO and mTz are Click-type reaction moieties that form a covalent binding interaction with each other.

Next, in FIG. 10C, the macromolecule 1015 is then denatured such that its monomers, residues, or moieties are more accessible to the fluidic medium that the macromolecule 1015 and the particle 1005 are within. Accordingly, the macromolecule 1015 unravels (or unfolds) and becomes more accessible to the fluidic medium and, therefore, to affinity reagents within the fluidic medium. This causes some of the attachment moieties of the macromolecule 1015 (e.g., attachment moieties 1025c and 1025d) to position closer to the surface of the particle 1005 than before the denaturing has occurred and attach with the attachment moieties of the particle 1005 (e.g., attachment moieties 1025c and 1025d in FIG. 10C). The binding interaction between TCO and mTz as the attachment moieties then prevents the macromolecule 1015 from reversing the unfolding and maintains improved accessibility to its monomers, residues, or moieties compared to the folded state depicted in FIG. 10A.

An attachment site or macromolecule, as set forth herein, may be provided with one or more spacing moieties. A spacing moiety may provide a separation distance between the entity to which the spacing moiety is attached and a second entity. A spacing moiety may comprise a rigid linker or a flexible linker. A rigid linker may comprise a moiety with decreased degrees of spatial freedom (e.g., with respect to rotational or translational motion), such as a double-stranded nucleic acid, an alkenyl moiety, or an alkynyl moiety. A flexible linker with increased degrees of spatial freedom (e.g., with respect to rotational or translational motion), such as a single-stranded nucleic acid, a peptide moiety, an alkyl moiety, or a synthetic polymer moiety.

A site or particle may comprise a plurality of attachment sites that are configured to couple a macromolecule to the site or particle. In some cases, a conformation of a macromolecule may be changed by dissociating binding between a complementary attachment moiety of a macromolecule and an attachment moiety of a first attachment site, and subsequently forming an association between the complementary attachment moiety and an attachment moiety of a second attachment site. Alternatively, a conformation of a macromolecule may be changed by dissociating binding between a first complementary attachment moiety of a macromolecule and an attachment moiety of a first attachment site, and forming an association between a second complementary attachment moiety and an attachment moiety of a second attachment site.

Accordingly, it may be advantageous to provide a plurality of attachment sites, in which individual attachment sites of the plurality of attachment sites comprise orthogonal attachment moieties. For example, attachment sites may comprise attachment oligonucleotides with differing nucleotide sequences. In another example, attachment sites may comprise orthogonal receptor-ligand binding pair components (e.g., streptavidin at a first attachment site, SpyCatcher at a second attachment site; a covalent attachment moiety at a first attachment site, a non-covalent attachment moiety at a second attachment site, etc.). Alternatively, it may be advantageous to provide a plurality of attachment sites, in which individual attachment sites of the plurality of attachment sites comprise identical attachment moieties. For example, two separate attachment sites may be provided with the same attachment oligonucleotide.

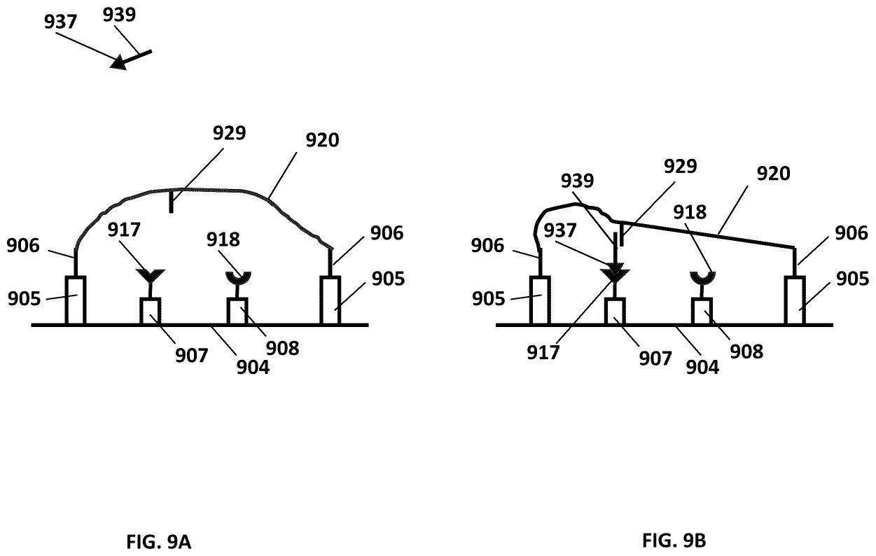

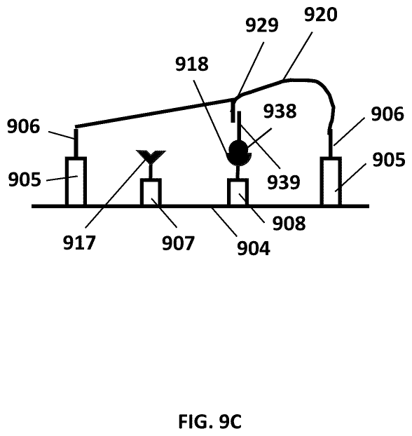

Nucleic acids may be particularly useful for altering a conformation of a macromolecule. FIGS. 9A-9C depict a system that utilizes a plurality of attachment sites having orthogonal attachment moieties to alter a conformation of a macromolecule. FIG. 9A depicts a macromolecule 920 that is attached to a site or particle 904 at two attachment sites 905 by attachment moieties 906. The site or particle 904 further comprises a first attachment site 907 having a first attachment moiety 917, and a second attachment site 908 having a second attachment moiety 918, in which the first attachment moiety 917 has a binding chemistry that is orthogonal to that of the second attachment moiety 918. The macromolecule 920 comprises or is attached to an oligonucleotide 929 at a residue, monomer, or moiety between the attachment points to attachment moieties 906. The site or particle 904 is contacted with a molecule comprising a complementary attachment moiety 937 that is complementary to first attachment moiety 917, and an oligonucleotide 939 comprising a nucleotide sequence that is complementary to oligonucleotide 929. FIG. 9B depicts a configuration in which the linking molecule has attached the macromolecule 920 to the first attachment site 907 by hybridization of oligonucleotide 939 to oligonucleotide 929, and coupling of complementary attachment moiety 937 to first attachment moiety 917. This configuration may constrain or extend the conformation of the macromolecule 920 between the first attachment site 907 and the rightmost attachment site 906. FIG. 9C depicts an alternative configuration, in which a different linking molecule has attached the macromolecule 920 to the second attachment site 908 by hybridization of oligonucleotide 939 to oligonucleotide 929, and coupling of complementary attachment moiety 938 to second attachment moiety 918.

The skilled person will readily recognize that choice of attachment moieties may be decided in part by orthogonality of dissociation of binding interactions. For example, in the system depicted in FIGS. 9A-9C, it may be preferable to utilize a nucleic acid that hybridize to an attachment moiety of the macromolecule and an attachment moiety of the site or particle for points of attachment that are intended to be disrupted (e.g., attachment to attachment sites 907 or 908), and a more stable attachment chemistry for attachment to attachment sites 905 (e.g., covalent binding, streptavidin-biotin, etc.). Accordingly, the nucleic acid attachment chemistry could be disrupted by heating or a chaotropic agent without dissociating the attachments of the more stable binding interactions.

In another aspect, provided herein is a method, comprising: a) contacting a plurality of binding reagents to a solid support, in which the solid support comprises a plurality of sites, optionally in which each site comprises a particle, in which the site or the particle attached thereto comprises a first attachment site and a second attachment site, and in which one and only one macromolecule is attached to the first attachment site and the second attachment site of the site or the particle attached thereto, b) coupling binding reagents to macromolecules at sites of the plurality of sites, and c) for each individual site, detecting presence or absence of a signal from a binding reagent of the plurality of binding reagents.

For some macromolecules, certain residues, monomers, or moieties may be inherently sequestered from contact with a fluid medium due to the properties of the fluid medium and the residues, monomers, or moieties. For example, hydrophobic amino acids may be sequestered within secondary or tertiary structures of proteins in an aqueous medium. Accordingly, the methods set forth herein for providing a macromolecule in an extended configuration may be useful for detecting the presence of said residues, monomers, or moieties. In some cases, a method may comprise coupling a binding reagent to an epitope containing an amino acid with a hydrophobic sidechain. In particular cases, a method may comprise coupling a binding reagent to an epitope containing two or more amino acids that each individually contain a hydrophobic sidechain.

It may be useful to provide avidity components at sites or particles attached thereto. An avidity component may comprise a binding entity that forms a weak binding interaction with a complementary avidity component. If a binding reagent is provided with a complementary avidity component, the weak binding interaction may be formed after the binding reagent binds to a binding target on a macromolecule. The effective dissociation rate of the binding reagent from the macromolecule may be decreased due to the presence of the binding interaction between the binding reagent and the macromolecule and the weak binding interaction between avidity components. In some cases, a site or a particle attached thereto may comprise a plurality of avidity components. In some cases, a binding reagent may comprise a plurality of complementary avidity components.

An avidity component may be provided in proximity to an attachment site. For example, an avidity component may be attached to a face of a particle adjacent to an attachment site on the face of the particle. In some cases, a site or a particle attached thereto may contain a first attachment site and a second attachment site, in which a first avidity component is coupled adjacent to the first attachment site, and a second avidity component is coupled adjacent to the second attachment site. The first avidity component may contain a moiety with the same binding specificity as the second avidity component. Alternatively, the first avidity component may contain a moiety with a differing binding specificity from the second avidity component.

The present disclosure provides compositions and methods for improving binding of analytes to affinity reagents by increasing avidity of the binding interaction. In particular embodiments, avidity between an analyte and affinity reagent can be increased by association of a first avidity component (hereinafter referred to as a “docker”) with the analyte and association of a second avidity component (hereinafter referred to as a “tether”) with the affinity reagent. The docker and tether recognize each other and can thus bind to each other. Avidity of the interaction between the affinity reagent and analyte is a function not only of recognition between the paratope and epitope, but also recognition between the docker and tether.

A docker can be associated with an analyte via covalent and/or non-covalent attachment of the docker to the analyte. Similarly, a tether can be associated with an affinity reagent via covalent and/or non-covalent attachment of the docker to the affinity reagent. Exemplary attachment chemistries include those set forth herein in the context of attaching analytes and affinity reagents to retaining components, addresses of an array, solid supports, labels, etc. In some configurations, a docker or tether can be attached to a particle (e.g., structured nucleic acid particle), unique identifier, address or solid support to which an analyte or affinity reagent, respectively, is attached.

Accordingly, the present disclosure provides a method of processing an analyte. The method can include the steps of (a) providing an analyte comprising an epitope and a docker; (b) providing an affinity reagent, wherein the affinity reagent comprises a paratope that recognizes the epitope and a tether that recognizes the docker; and (c) contacting the analyte with the affinity reagent, whereby the affinity reagent associates with the analyte via binding of the paratope to the epitope and via binding of the tether to the docker. Optionally, the method further includes a step of detecting association of the affinity reagent with the analyte, thereby identifying the analyte. In another option, the analyte is present in a sample including other analytes and the method further includes a step of separating the analyte from the other analytes via the association of the affinity reagent with the analyte.

The compositions and methods of the present disclosure are particularly well suited for detecting analytes using affinity reagents in non-equilibrium conditions. A typical binding assay employs an excess amount of affinity reagent and immobilized analytes to drive formation of an immobilized complex between the affinity reagent and analyte. In some assays the excess labeled affinity reagent in solution produces unwanted background that overwhelms signal produced by immobilized complexes. Removal of excess affinity reagents from solution creates a non-equilibrium condition that drives affinity reagents to dissociate from the immobilized analytes. The use of tethers and dockers can increase the half-life of the complexes under non-equilibrium conditions, thereby improving detectability of analyte-affinity reagent complexes.

A variety of different types of dockers and tethers can be employed to increase avidity of binding between an analyte and affinity reagent. The type of docker and tether that is to be used in combination with a particular analyte and affinity reagent pair can be selected based on known or expected affinity of the affinity reagent for the analyte. For example, a method that employs a first affinity reagent having relatively strong affinity for a particular analyte can utilize a docker and tether pair having relatively weak affinity, whereas a method that employs a second affinity reagent having weaker affinity for the analyte can utilize a docker and tether pair having higher affinity compared to the pair used for the first affinity reagent. Accordingly, the probability of forming a complex and duration of the complex can be tuned by appropriate choice of docker type and tether type.

A docker can be any molecule or moiety that is capable of binding to a tether and a tether can be any molecule or moiety that is capable of binding to a docker. A particularly useful docker or tether is a nucleic acid strand having a nucleotide sequence that complements a nucleotide sequences of a tether or docker, respectively. A nucleic acid strand that is used as a docker or tether can include a sequence of at least 3, 4, 5, 6, 7, 8, 9, 10, 12, 14, 16, 18, 20, 25 or more nucleotides. Alternatively or additionally, a nucleic acid strand that is used as a docker or tether can include a sequence of at most 25, 20, 18, 16, 14, 12, 10, 9, 8, 7, 6, 5, 4, 3 or fewer nucleotides. Other useful dockers or tethers include, for example, a receptor that recognizes a ligand, a ligand that recognizes a receptor, an affinity reagent that recognizes an analyte, an analyte that recognizes an affinity reagent, a paratope that recognizes an epitope, an epitope that recognizes a paratope, or a reactive moiety that forms a covalent bond with another reactive moiety. Exemplary dockers or tethers include, but are not limited to, an antibody, Fab′ fragment, F(ab′)2 fragment, single-chain variable fragments, di-scFv, tri-scFv, microantibody, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag. In some configurations, a docker or tether can be a protein that recognizes a nucleic acid sequence such as a DNA binding protein or RNA binding protein. Exemplary nucleic acid-binding proteins, which can be used as dockers or tethers, and the nucleic acid moieties to which they bind, which can be used as tethers or dockers, respectively, include a Toll-Like Receptor (TLR) which binds to DNA having a CpG moiety, transcription factor which binds to a specific nucleic acid sequence, or histone protein(s) which binds to DNA. Further examples are provided in the Eukaryotic nucleic acid binding protein database (ENPD). See Leung et al. Nucleic Acids Res. 47 (Database issue): D322-D329 (2019), which is incorporated herein by reference.

A further variable that can be employed to tune binding between an analyte and affinity reagent is the number of dockers associated with the analyte and/or the number of tethers associated with the affinity reagent. For example, a method that employs a first affinity reagent having relatively strong affinity for an analyte can utilize a relatively low number of docker-tether pairs, whereas a method that employs a second affinity reagent having weaker affinity for the analyte can utilize a greater number of docker-tether pairs compared to the number(s) used for the first affinity reagent.

An analyte can be associated with a single docker or, alternatively, with a plurality of dockers. For example, an analyte can be associated with at least 2, 3, 4, 5, 6, 7, 8, 9, 10, 15, 20, 25, 50 or more dockers. Alternatively or additionally, an analyte can be associated with at most 50, 25, 20, 15, 10, 9, 8, 7, 6, 5, 4, 3, 2 or fewer dockers. The dockers can be substantially identical to each other, thereby recognizing the same tethers. Alternatively, a plurality of dockers can include dockers that differ from each other. In some cases, the different dockers will recognize different tethers. It is also possible for the different dockers to recognize the same tethers. In some configurations, an analyte and the docker with which it is associated will have binding characteristics that are orthogonal to each other. As such, a paratope of an affinity reagent that recognizes or binds to the analyte will not recognize or bind to the docker, and a tether that recognizes or binds to the docker will not recognize or bind to the analyte.

An affinity reagent can be associated with a plurality of tethers. For example, an affinity reagent can be associated with at least 2, 3, 4, 5, 6, 7, 8, 9, 10, 15, 20, 25, 50 or more tethers. Alternatively or additionally, an affinity reagent can be associated with at most 50, 25, 20, 15, 10, 9, 8, 7, 6, 5, 4, 3, 2 or fewer tethers. The tethers can be substantially identical to each other, thereby recognizing the same dockers. Alternatively, a plurality of tethers can include tethers that differ from each other. In some cases, the different tethers will recognize different dockers. It is also possible for the different tethers to recognize the same dockers. In some configurations, an affinity reagent and the tether with which it is associated will have orthogonal binding recognition. As such, an analyte that recognizes or binds to a paratope of the affinity reagent will not recognize or bind to the tether, and a docker that recognizes or binds to the tether will not recognize or bind to the paratope.

Of course, a binding event can be tuned via a combination of the number and type of docker-tether pairs used. This can be illustrated in the context of nucleic acid dockers and tethers having complementary nucleotide sequences. For example, the maintenance of a complex between an analyte and affinity reagent can be increased by increasing the number of dockers and tethers present in the complex and also by increasing the avidity of each docker for its complementary tether. The avidity of binding between a nucleic acid docker and tether can be increased, for example, by increasing the length of the complementary sequences, increasing the GC content of the complementary sequences, or otherwise increasing the melting temperature (Tm) of the duplex formed by the complementary sequences. The length of the complementary sequences can be at least 3, 4, 5, 6, 7, 8, 9, 10, 12, 14, 16, 18, 20, 25 or more nucleotides. Alternatively or additionally, the length of the complementary sequences can be at most 25, 20, 18, 16, 14, 12, 10, 9, 8, 7, 6, 5, 4, 3 or fewer nucleotides. The GC content of the complementary sequences can be at least 25%, 40%, 50%, 60%, 75%, or higher. Alternatively or additionally, the GC content of the complementary sequences can be at most 75%, 60%, 50%, 40%, 25% or lower.

Multiplex methods, in which a plurality of different analytes is processed in parallel, can employ universal dockers. The dockers are referred to as ‘universal’ because they are identical with respect to structural features that interact with tethers. For example, an array can include a plurality of addresses, each of the addresses being attached to an analyte that differs from other analytes in the array and each of the addresses being attached to a docker that is the same as other dockers in the array. A plurality of different analytes that are associated with universal dockers can be contacted with a plurality of different affinity reagents that are associated with tethers. Some or all the different affinity reagents can have the same tether structure. As such, the avidity effect of the dockers and tethers can be substantially uniform.

Methods that employ multiple different affinity reagents can employ universal tethers. The tethers are referred to as ‘universal’ because they are identical with respect to structural features that interact with dockers. For example, an array of analytes can be contacted with a plurality of different affinity reagents, each of the affinity reagents having a paratope that differs from other affinity reagents in the plurality and each of the affinity reagents being attached to a tether that is the same as other tethers in the plurality. The different affinity reagents can be present in a mixture that is simultaneously in contact with the array or, alternatively, the different affinity reagents can be serially contacted with the array.

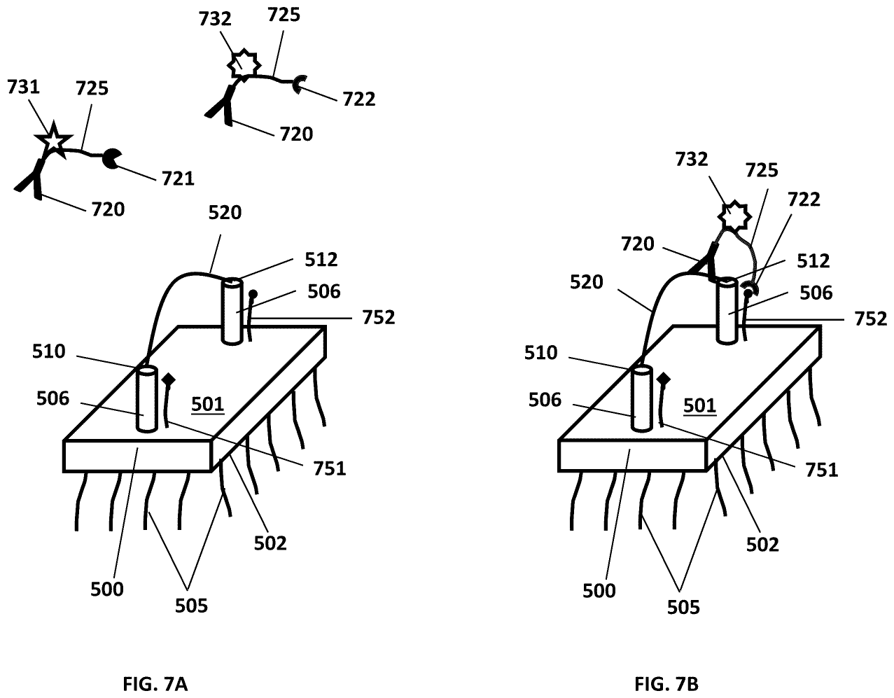

FIGS. 7A-7B illustrate use of avidity components to facilitate detection of a binding target (e.g., an epitope, a tag) of a macromolecule. As shown in FIG. 7A, a macromolecule 520 that is coupled to a first attachment site 510 and a second attachment site 512 is provided in a similar fashion as described in FIGS. 5A-5F. A first avidity component 751 is coupled to the first face 501 of the particle 500 adjacent to the first attachment site 510. A second avidity component 752 is coupled to the first face 501 of the particle 500 adjacent to the second attachment site 512. The solid support containing the macromolecule 520 is contacted with a plurality of binding reagents. Each binding reagent comprises an affinity agent 720 (e.g., an antibody, an antibody fragment, an aptamer, a peptamer, etc.). A binding reagent of the plurality of binding reagents further comprises a first complementary avidity component 721 that is configured to bind to the first avidity component 751, a first detectable label 731, and optionally a linking moiety 725 (e.g., a particle, a polymeric chain) that couples together the affinity agent 720, the detectable label 731, and the first complementary avidity component 721. A second binding reagent of the plurality of binding reagents further comprises a second complementary avidity component 722 that is configured to bind to the second avidity component 752, a second detectable label 732, and optionally a linking moiety 725 that couples together the affinity agent 720, the detectable label 732, and the second complementary avidity component 722. FIG. 7B depicts a configuration after an affinity reagent 720 has bound an epitope of the macromolecule 520. Because the binding target of the affinity agent 720 is located nearer to the second attachment site, coupling of a binding reagent containing a complementary second avidity component 722 may be preferred due to the presence of two binding interactions facilitating association or inhibiting dissociation of the binding reagent at the binding target. Accordingly, detection of a signal from the second detectable label 732 may provide a spatial position of the binding target within the macromolecule 520 relative to the residue, monomer, or moiety of the macromolecule attached to the second attachment site 512. It will be readily recognized that detection can also be multiplexed by utilizing two or more binding reagents that differ with respect to binding target.

Certain assays for interrogating macromolecules may utilize binding reagents that form covalent or non-covalent binding interactions with macromolecules. A binding reagent can include any suitable reagent that: i) forms a binding interaction with a macromolecule, and ii) produces a detectable signal when bound to the macromolecule, thereby facilitating detection of the macromolecule-binding reagent complex at an address containing the macromolecule. A useful configuration of a binding reagent comprises an affinity reagent (e.g., an antibody, an antibody fragment, an aptamer, a peptamer) coupled to a detectable label (e.g., a fluorescent or luminescent moiety). A binding reagent may further comprise a complementary avidity component, as set forth herein. In some cases, a binding reagent may comprise a plurality of affinity reagents and/or detectable labels. Binding reagents comprising a plurality of affinity reagents and/or detectable labels are described in U.S. Pat. No. 11,692,217 which is herein incorporated by reference in its entirety. In some cases, components of a binding reagent (e.g., an affinity reagent, a detectable label, an avidity component) may be joined by a linking moiety (e.g., a particle, a polymeric chain, a branched or dendrimeric polymer, etc.). Nucleic acid nanoparticles may be useful for binding reagents due to the ability to attach various components of a binding reagent at specific tunable locations of the nanoparticles. Useful binding reagent compositions are disclosed in U.S. Pat. No. 11,692,217 and U.S. patent application Ser. No. 18/438,973, each of which is herein incorporated by reference in its entirety.

A method, as set forth herein, may involve one or more steps, including: i) contacting a reagent to a solid support optionally in the presence of a reagent association fluid medium, thereby associating the reagent (e.g., a reactive agent, an affinity agent, a binding reagent, etc.) to a macromolecule or an array thereof, as set forth herein; ii) after an association step, rinsing unassociated reagents from the solid support utilizing a rinsing medium; iii) detecting presence or absence of association of the reagent to the macromolecule or macromolecules of the array optionally in the presence of a detection fluid medium; iv) optionally dissociating reagents from the macromolecules or macromolecules of the array optionally in the presence of a reagent dissociation fluid medium; v) after a dissociation step, rinsing unassociated reagents from the solid support utilizing a rinsing medium; and optionally repeating one or more of steps i)-v) for at least one additional cycle comprising one or more repeated steps (e.g., at least about 2, 3, 4, 5, 10, 20, 50, 100, 150, 200, 300, 500, 1000 or more than 1000 cycles).

The conformation of a macromolecule may affect the ability of a binding reagent to bind to the macromolecule. See, for example, Forsstrom, B., et al. “Dissecting Antibodies with Regards to Linear and Conformational Epitopes.” PLOS One (2015), which is herein incorporated by reference in its entirety. For example, a binding reagent may have a binding specificity for a conformational epitope of a polypeptide (i.e., an epitope formed by a secondary or tertiary structure of the polypeptide that brings non-consecutive amino acids into close proximity). Accordingly, disruption of the conformational epitope may inhibit an ability of the binding reagent to bind to the polypeptide. Likewise, a binding reagent may have a binding specificity for a linear epitope of a polypeptide (i.e., an epitope formed by a consecutive sequence of amino acids). Accordingly, formation of a secondary or tertiary structure of the polypeptide may inhibit an ability of the binding reagent to bind to the polypeptide. A method may comprise repeatedly measuring presence or absence of association of a binding reagent to a macromolecule, in which a conformation of the macromolecule is altered by a method set forth herein between association detections. A method may comprise repeatedly measuring presence or absence of association of a binding reagent to a macromolecule, in which a conformation of the macromolecule is altered by a method set forth herein during a single association event.

A method may comprise altering the extension of a macromolecule while detecting the association of a binding reagent to the macromolecule. Extension of the macromolecule may inhibit binding of the binding reagent or may facilitate binding of the binding reagent. For example, an ATP-dependent analyte-binding group may be utilized to increase the extension of a polypeptide by serially providing ATP to the analyte-binding group. A sequence of detection events may occur to identify when a polypeptide conformation has been disrupted, thereby dissociating a binding reagent from a conformational epitope of the polypeptide conformation.

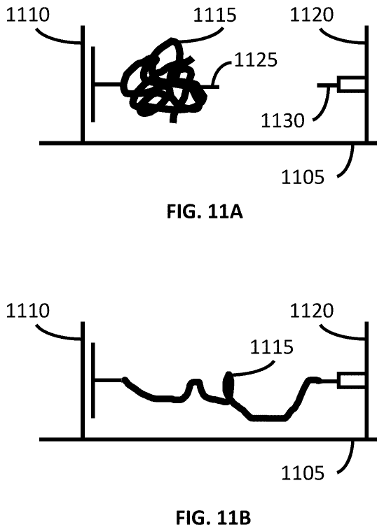

FIGS. 11A and 11B depict an example of unfolding a macromolecule via tackboard pinning. In FIG. 11A, macromolecule 1115 is attached to a particle (e.g., a nucleic acid particle like the examples of FIGS. 5A, 5F, and elsewhere described herein) to form an analyte-particle complex. However, rather than having the bottom surface of the particle resting upon a surface 1105 of a flow cell such that the macromolecule 1115 is positioned upwards and away from the surface 1105, the particle is instead attached in a perpendicular fashion to a landing site 1110 and positioned towards a tackboard 1120. That is, macromolecule 1115 is positioned between two different structures attached to the surface: the tackboard 1120 and the landing site 1110. Moreover, the macromolecule (or analyte) is positioned distal to the landing site 1110 in comparison to the other surface of the particle which is positioned proximate to the landing site 1110.

Further, the macromolecule 1115 includes one or more attachment moieties 1125. Similarly, the tackboard 1120 includes one or more attachment moieties 1130. The attachment of the particle with the macromolecule 1115 may be done with one or more nucleic acid strands (attached to the other surface of the particle facing away from the macromolecule 1115 and towards landing site 1110) that comprise a nucleic acid sequence that is complementary to a nucleic acid sequence of site-coupled nucleic acid strands at the landing site 1110, via Click-chemistry, or any other attachment techniques as described herein.

Next, in FIG. 11B, the macromolecule 1115 is denatured to disturb its spatial conformation and, therefore, unfolds such that its one or more attachment moieties 1125 are positioned closer to the one or more attachment moieties 1130 of the tackboard 1120. This causes an attachment like those described elsewhere herein (e.g., a Click-chemistry such as mTz-TCO). This also causes the macromolecule 1115 to be stretched or more linearized along an axis between the landing site 1110 and the tackboard 1120 such that its monomers, residues, or moieties are more accessible to the fluidic medium and accessible to reagents disposed therein.

In one example, the structure of the landing site 1110 and the tackboard 1120 in FIGS. 11A and 11B may be defined by a well (or nanowell) in which the particle with the macromolecule 1115 flows through a flow channel above the well and then settles down through an opening of a chamber defined by the well's interface or opening with the flow channel and the structural material of the flow cell serving as “walls” or a bottom surface enclosing all other dimensions other than the opening. In another example, the landing site 1110 and the tackboard 1105 may be structures positioned on a planar surface of a flow cell to serve as the bottom surface but without other material serving as a “wall” of a well to enclose the macromolecule 1115 other than the opening.

The landing site 1110 and the tackboard 1120 may be composed of any of the molecules or materials described herein, including nucleic acid (e.g., via nucleic acid origami, or DNA origami), dendrimers, etc. that include the corresponding attachment sites to enable the binding interaction, attachment, or coupling to facilitate the extension of the macromolecule from the landing site 1110 to the tackboard 1120.

In some implementations, the macromolecule 1115 may be initially attached with the particle and attachment moiety 1125 in solution to form the analyte-particle complex. Next, the macromolecule 1115 attached to the particle may be attached with the landing site 1110 in solution. Alternatively, each of the macromolecule 1115, the attachment moiety 1125, and the particle may be formed in a mixture together in solution.

Subsequently, the entire complex of the landing site 1110 with the particle and the macromolecule 1115 may be introduced into the flow cell and the landing site 1110 may attach at specific, optically resolvable locations on the flow cell. For example, the landing site 1110 may have one or more strands of nucleic acid that are complementary to one or more strands of nucleic acid that are deposited upon specific locations of the flow cell. In another example, complementary Click-chemistries may be used to facilitate the attachment of the landing site 1110 to the specific locations of the flow cell. Alternatively, one or more parts (including all) of the complex may be formed while in the flow cell.

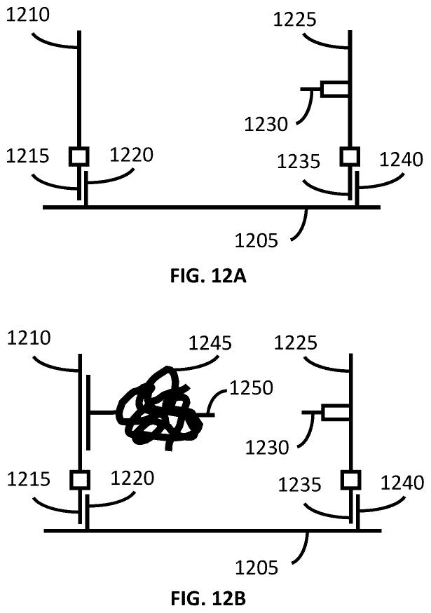

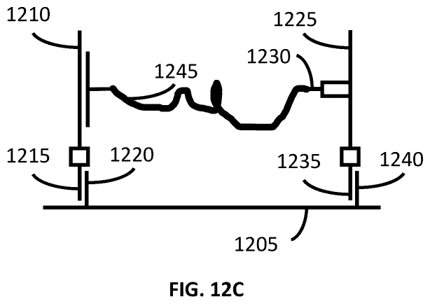

In another example, the landing sites and tackboards may flow in a flow channel and then deposit upon the surface to form the sites for the macromolecules to be stretched or linearized. FIG. 12A depicts a landing site 1210 with an attachment site 1215 that attaches with attachment site 1220 of the surface 1205, which may be the surface of a flow cell, chip, or other substrate. Likewise, a tackboard 1225 is also depicted as attached and immobilized upon the surface 1205 via the attachment between attachment moieties 1235 and 1240. The attachment moieties can be any of the techniques described herein, including Click-chemistries or nucleic acid sequences with complementary sequences such that the attachment moiety 1215 attaches with the attachment moiety 1220 but fails to attach with the attachment moiety 1240. Likewise, if using nucleic acid strands, the attachment moiety 1235 might have a complementary nucleic acid sequence to the attachment moiety 1240 that is different than the nucleic acid sequence of the attachment moiety 1220. Thus, by having different complementary nucleic acid sequences or Click-chemistries for the attachment moieties 1220 and 1240, the landing site 1210 and the tackboard 1225 may be assembled in the expected locations upon surface 1205. A denaturant may then be applied, resulting in the macromolecule 1245 altering its spatial conformation and attaching with the tackboard 1225 as previously discussed.

The techniques with depositing the landing sites and tackboards can be performed in other ways. For example, a bridge nucleic acid strand may be used to hybridize and therefore attach with both an attachment moiety on the surface of the flow cell as well as the attachment moiety on the tackboard. A bridge nucleic acid strand may disassociate (e.g., via toehold mediated strand displacement) to reverse the attachment. Bridge nucleic acid strands are described in U.S. patent application Ser. No. 18/813,886, titled Compositions and Methods for Detecting Binding Interactions Under Equilibrium or Non-Equilibrium Conditions, filed on Dec. 3, 2024, which is incorporated herein in its entirety and for all purposes. As another example, the strands (or attachment moieties) may be DNA/RNA duplexes, and RNA may be used for the bridge oligo strand. The RNA-based bridge strand may then be removed via an application of Rnase H.

As a result, the bridge nucleic acid strand may have a portion with a nucleotide sequence that does not hybridize with the tackboard's 1225 attachment moiety 1240 but rather hybridizes with the attachment moiety 1220 on the surface of the flow cell 1205. Additionally, the bridge nucleic acid strand may have another portion with a nucleotide sequence that hybridizes with the tackboard's 1225 attachment moiety's 1235 nucleotide sequence rather than that of the surface.

In some implementations, a macromolecule may be unfolded at different lengths while attached to a single position (or attachment site or attachment moiety) on the surface using the techniques described herein. Affinity reagents, or probes, may then be iteratively applied to a macromolecule as it is stretched to different lengths and, therefore, the macromolecule's monomers, residues, or moieties may be exposed to the affinity reagents as the monomers, residues, or moieties are increasingly made more accessible to the affinity reagents in the fluidic medium.

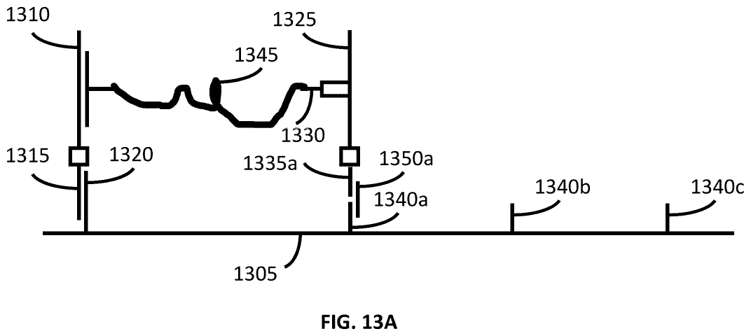

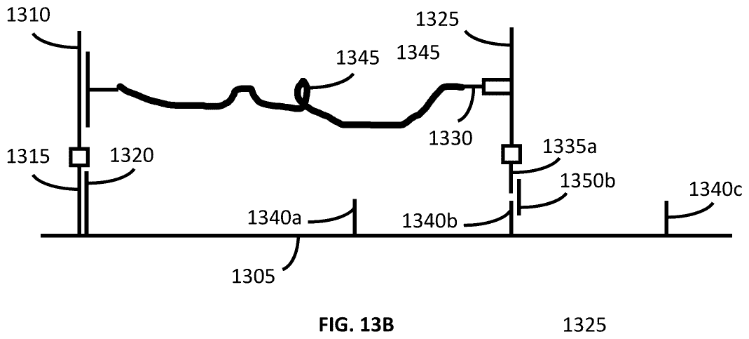

FIGS. 13A and 13B depict an example of unfolding a macromolecule at different lengths. In FIG. 13A, landing site 1310 is attached (or immobilized) at an attachment site upon the surface 1305 of a flow cell via an attachment moiety 1320 (e.g., nucleic acid strand) which hybridizes with attachment moiety 1315 (e.g., a nucleic acid strand with a sequence that is complementary to the sequence of attachment moiety 1320). A tackboard 1325 also hybridizes to a bridge nucleic acid strand 1350a. The bridge nucleic acid strand 1350a also hybridizes with attachment moiety 1340a. A portion of the sequence of the bridge nucleic acid strand 1350a is complementary to the sequence of the attachment moiety 1340a. However, the bridge nucleic acid strand 1350a does not have a sequence that is complementary to the sequences of the attachment moieties 1340b or 1340c. As a result, the bridge nucleic acid strand 1325 immobilizes the tackboard 1325 at one of three locations that are available for a tackboard to be immobilized upon.

Because each of the three attachment moieties 1340a, 1340b, and 1340c are at different distances from the landing site 1310 that is attached at the position of the attachment moiety 1320, the macromolecule 1345 may be stretched or linearized to different lengths in accordance with the different distances of the attachment sites if the macromolecule 1345 is denatured in accordance with the techniques described herein. For example, the attachment moieties 1340a, 1340b, and 1340c are positioned such that they are spaced 50 nanometers (nm) apart from each other. Thus, depending on where the tackboard 1325 is immobilized, the macromolecule 1345 may be extended to different possible lengths from 50 nm, 100 nm, and 150 nm away from the attachment moiety 1320.

The 50 nm, 100 nm, and 150 nm described herein are merely examples and any lengths may be used. Moreover, the distance between each of the attachment moieties need not be uniform. For example, the distance between a first attachment moiety and a second attachment moiety may be 60 nm, and the distance between the second attachment moiety and a third attachment moiety may be 120 nm.

In the example of FIG. 13A, the landing site 1310 is attached at the attachment site 1320 and the tackboard 1325 is attached at the landing site 1340a. The macromolecule 1345 that is attached with the particle forming the analyte-particle complex (which, in turn, is attached with the landing site 1310) is then denatured, and then attaches with the attachment moiety 1330 of the tackboard 1325, as previously discussed. The macromolecule 1345 can then be iteratively probed using affinity agents such that binding measurements of the macromolecule 1345 to those affinity reagents can be determined and then used to characterize the macromolecule. For example, the macromolecule 1325 identification or quantity (i.e., how many of the species of macromolecule 1345 are from a sample that is applied to the flow cell) can be determined. As another example, the PTMs of the macromolecule 1325 may be identified or quantified. As another example, isoforms of the macromolecule 1325 may be identified or quantified. Thus, any individual or combination characteristics of the macromolecule 1325 may be characterized, including one or more of its: identity, quantity, PTMs, and/or isoforms. PTMs and/or isoforms of a protein may also be referred to as “proteoforms”. The characterization of the proteins may be performed via a “protein identification by short epitope mapping” (or “prism”) approach which is described in detail in U.S. Pat. Nos. 10,473,654B1, 11,545,234B1, and Eggertson, et al. bioRxiv, https://doi.org/10.1101/2021.10.11.463967, each of which is incorporated by reference in their entirety herein.

In FIG. 13B, the bridge nucleic acid strand 1350a is disassociated and a bridge nucleic acid strand 1350b includes one region with a sequence that is complementary to the sequence of the attachment moiety 1335a and another region with a sequence that is complementary to the sequence of the attachment moiety 1340b, resulting in the tackboard 1325 at a different position than as depicted in FIG. 13A. This causes the macromolecule 1345 to be extended in length an increased amount and increasing the accessibility of its monomers, residues, or moieties. The macromolecule 1345 can then be iteratively probed at this second, longer linearization using affinity agents such that binding measurements of the macromolecule 1345 to the affinity reagents can be determined. The results of the binding measurements may different due to the increased accessibility, therefore facilitating improved characterization of the macromolecule 1345.

In some implementations, affinity reagents may first be applied to a macromolecule when it has not been denatured. After collecting binding measurements, the macromolecule may then be linearized, and the affinity reagents may be applied a second time to collect binding measurements with the macromolecule in two different “levels” of accessibility. The macromolecule may be subject to additional levels of accessibility by additional linearization to different lengths.

In some implementations, the macromolecule may be subject to an enzyme to remove post-translational modifications (PTMs). For example, a protein in a non-denatured spatial conformation may first be subject to the binding measurements via iterative application of affinity reagents. Later, the protein may be denatured and linearized at a first length and then a deglycosylation enzyme may be applied to remove some of the PTMs (e.g., glycans) that are now accessible following the linearization. New binding measurements may be obtained, and then the protein may be linearized at a second length that is different than the first length. The deglycosylation enzyme may be applied again to now remove some of the PTMs that were previous inaccessible at the first length of linearization but are now accessible at the second length of linearization. Next, binding measurements may be obtained with the protein linearized at the second length. The binding measurements obtained at different linearization as well as the non-denatured (or minimally or less denatured) protein may then be utilized in the decoding techniques previously described to characterize the proteins on the flow cell, including identifying or quantifying the proteins. Other types of enzymes that can be applied to remove PTMs include enzymes that dephosphorylate (e.g., remove a phosphate group), de-ubiquitinate (e.g., remove ubiquitin), deacetylation (e.g., remove an acetyl group), or demethylation (e.g., remove a methyl group).

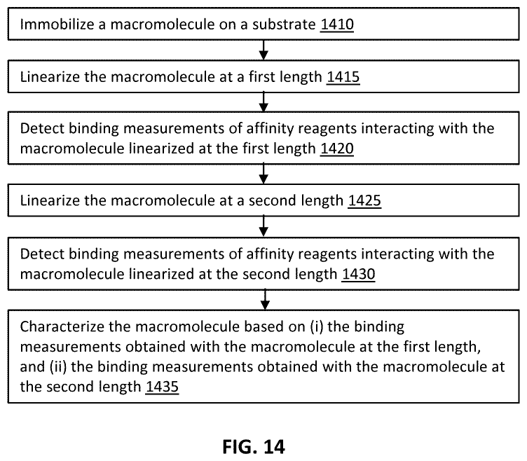

FIG. 14 depicts flow diagram for probing a macromolecule with an unfolded spatial conformation at different lengths. In FIG. 14, a macromolecule may be immobilized on a substrate (1410). For example, the macromolecule 1345 in FIG. 13A may be immobilized via the particle and the landing site 1310 immobilized at the attachment moiety 1320.

Next, the macromolecule may be linearized at a first length (145). For example, in FIG. 13A, the macromolecule 1345 is linearized based on the tackboard 1325 positioned at attachment moiety 1340a.

Binding measurements of affinity reagents interacting with the macromolecule linearized at the first length may be detected (1420). For example, the macromolecule 1345 may be iteratively probed with different affinity reagents.

The macromolecule may then be linearized at a second length that is different than the first length (1425). For example, in FIG. 13B, the macromolecule 1345 is linearized longer due to the tackboard 1325 positioned at attachment moiety 1340b.

Binding measurements of affinity reagents interacting with the macromolecule linearized at the second length may then be performed (1430). For example, the macromolecule 1345 linearized in FIG. 13B is subjected to affinity reagents that may be able to contact additional monomers, residues, or moieties that were not accessible when the linearization was shorter as depicted in FIG. 13A.

As a result, the macromolecule is characterized based on (i) the binding measurements obtained with the macromolecule at the first length, and (ii) the biding measurements obtained with the macromolecule at the second length (1435). For example, the identity, quantity, PTMs, or isoforms of the macromolecule by be determined.

Many of the techniques describe denaturing a macromolecule that is a protein. Denaturing may be performed via introduction of a denaturant into the fluidic medium, introduction of enzymes or unfoldases into the fluidic medium to “open up” proteins to increase accessibility to its residues, introduction of other proteins into the fluidic medium and causing protein-to-protein interactions that also open up the proteins-of-interest that are immobilized on the surface, or adjusting characteristics of the fluidic medium including its temperature.

Many of the techniques described herein linearize a macromolecule to increase accessibility of monomers, residues, or moieties to the surrounding fluidic medium. However, any of the denaturing techniques may also be performed in place of linearizing the macromolecule. For example, different denaturing techniques may be applied to change the spatial conformation of a protein and iterative probing may be applied while the protein is in different spatial conformations due to the different denaturing techniques. Thus, the identity, quantity, PTMs, or isoforms of the proteins may be characterized as previously discussed.

Certain macromolecules may comprise residues or sequences of residues that are hydrophobic. For example, polypeptide molecules may comprise hydrophobic amino acids that are folded into a hydrophobic core of the molecule when contacted to an aqueous medium. Depending upon the size and function of a polypeptide, certain polypeptide molecules may have a greater amount of hydrophobic residues. For example, a small globular protein will likely have fewer hydrophobic residues than a large membrane protein. Accordingly, the extent of hydrophobicity may be identifiable by a method of altering macromolecule conformation set forth herein. In some cases, a macromolecule may be extended into a partially- or fully-denatured state, thereby exposing one or more regions of hydrophobicity of the macromolecule to an aqueous medium. A method may further comprise contacting the macromolecule with a hydrophobic binding reagent, thereby associating the hydrophobic binding entity to a hydrophobic region of the macromolecule. A hydrophobic binding reagent could comprise a binding reagent, as set forth herein, containing one or more hydrophobic moieties (e.g., lipids, alkyl moieties, etc.). It may be useful to attach the hydrophobic moieties to a hydrophilic moiety such as a nucleic acid nanoparticle or a polymer particle (e.g., a PEG molecule) to improve dispersion of the binding reagent in an aqueous medium. Alternatively, certain fluorescent molecules that are useful for digital scanning fluorimetry techniques may be useful for binding to partially- or fully-denatured proteins, such as SYPRO Orange, N-[4-(7-diethylamino-4-methyl-3-coumarin)phenyl]maleimide, and 4-(dicyanovinyl) julolidine.

An advantage of the present systems and/or methods may be the ability to maintain a macromolecule in an extended or partially-denatured state in the absence of a denaturing agent or chaotrope. Accordingly, certain fluid media (e.g., a reagent association medium, a reagent dissociation medium, a detection medium, a rinsing medium, etc.) utilized during a step of a method set forth herein may be substantially devoid of a denaturing agent or chaotrope. Alternatively, a fluid medium utilized during a step of a method set forth herein may comprise a lower concentration of a denaturing agent or a chaotrope of no more than a concentration of the denaturing agent or chaotrope present in a fluid medium utilized to prepare a macromolecule and/or attach the macromolecule to a site or a particle.

In some cases, a macromolecule may be provided to a system or method set forth herein in a partially- or fully-denatured conformation. In some cases, a macromolecule may be provided to a system or method set forth herein in a fluidic medium that comprises a denaturing species and/or a surfactant species, thereby maintaining the macromolecule in a partially- or fully-denatured conformation. In some cases, a method may comprise a step of partially or fully denaturing a macromolecule, then attaching the macromolecule to a site or particle by a method set forth herein. In other cases, a method may comprise a step of attaching the macromolecule to a site or particle by a method set forth herein, then partially or fully denaturing the macromolecule.

### Assays for Analyte Analysis

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as formation of a nucleic acid tag, that is detected after the complex has been formed and in some cases after the complex has been dissociated.

Many protein detection methods, such as enzyme linked immunosorbent assay (ELISA), achieve high-confidence characterization of one or more protein in a sample by exploiting high specificity binding of antibodies, aptamers or other binding agents to the protein(s). ELISA is generally carried out at low plex scale (e.g., from one to a hundred different proteins detected in parallel or in succession) but can be used at higher plexity is some situations for which specialized hardware or reagents are available. ELISA methods can be carried out by detecting immobilized binding agents and/or proteins in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX, LUMINEX® 100/200 or FEXMAP 3D®.

Other detection methods that can also be used, for example at low plex scale, include procedures that employ SOMAmer reagents and SOMAscan assays commercialized by Soma Logic (Boulder, CO). In one configuration, a sample is contacted with aptamers that are capable of binding proteins with specificity for the amino acid sequence of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g., the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in U.S. Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g., amount or concentration), chemical reactivity, molecular structure, structural integrity (e.g., full length or fragmented), maturation state (e.g., presence or absence of pre- or pro-sequence in a protein), location (e.g., in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g., amino acid sequence length, overall charge or overall pKa for a protein) or common moiety (e.g., a short primary sequence motif or post-translational modification for a protein). An analyte can be characterized with regard to a relatively specific characteristic such as a unique amino acid sequence (e.g., for the full length of the protein or a motif), an RNA or DNA sequence that encodes a protein (e.g., for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers are expected to occur in a substantial number of different proteins in a typical proteome. Alternatively or additionally, a given promiscuous affinity reagent may recognize multiple different epitopes (e.g., epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g., positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. It will be recognized that methods set forth herein that are utilized to decode extant proteins may be useful for other analyte identification assays, provided said analyte identification assays provide a binding profile that can be decoded.

In some detection assays, a protein can be cyclically modified and the modified products from individual cycles can be detected. For example, a protein can be sequenced by a sequential process in which each cycle includes steps of detecting the protein and removing one or more terminal amino acids from the protein to produce a shortened protein. The shortened protein is then subjected to subsequent cycles. Optionally, a protein sequencing method can include steps of adding a label to the protein, for example, at the amino terminal amino acid or at the carboxy terminal amino acid. In particular configurations, a method a protein sequencing method can include steps of (i) removing a terminal amino acid from the protein, thereby forming a truncated protein; (ii) detecting a change in signal from the truncated protein, for example, in comparison to the protein prior to truncation; and (iii) identifying the type of amino acid that was removed in step (i) based on the change detected in step (ii). The terminal amino acid can be removed, for example, by removal of one or more amino acids from the amino terminus or carboxyl terminus of the protein. Steps (i) through (iii) can be repeated to produce a series of signal changes that is indicative of the sequence for the protein.

In a first configuration of a protein sequencing method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. Nos. 9,625,469 or 10,545,153, each of which is incorporated herein by reference. Methods and apparatus under development by Erisyon, Inc. (Austin, TX) may also be useful for sequencing, or otherwise detecting, proteins.

In a second configuration of a cyclical protein detection method, a terminal amino acid of a protein can be recognized by an affinity agent that is specific for the terminal amino acid, specific for a labeled terminal amino acid (e.g., the affinity agent can recognize the label alone or in combination with the side chain of a particular type of amino acid). The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) or Standard BioTools (e.g., technology developed by SomaLogic or Palamedrix) may also be useful for detecting proteins.

Cyclical removal of terminal amino acids from a protein can be carried out using an Edman-type sequencing reaction. In some configurations, an Edman-type sequencing reaction can involve reaction of a phenyl isothiocyanate with an N-terminal amino group of a protein under mildly alkaline conditions (e.g., about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. https://doi.org/10.1007/978-1-4899-1031-8_8).

Non-limiting examples of functional groups for substituted phenyl isothiocyanate may include ligands (e.g., biotin and biotin analogs) for known receptors, labels such as luminophores, or reactive groups such as click functionalities (e.g., compositions having an azide or acetylene moiety). The functional group may be a DNA, RNA, peptide or small molecule barcode or other tag which may be further processed and/or detected.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid in the presence of the nucleic acid tag, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. The extending of a primer can be carried out, for example, by polymerase-based extension of the primer, using the nucleic acid tag as a template. Alternatively, the extending of a primer can be carried out, for example, by ligase- or chemical-based ligation of the primer to a nucleic acid that is hybridized to the nucleic acid tag. The nucleic acid tag can be detected via hybridization to nucleic acid probes (e.g., in an array), amplification-based detections (e.g., PCR-based detection, or rolling circle amplification-based detection) or nuclei acid sequencing (e.g., cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as other proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

In some configurations of the apparatus and methods set forth herein, one or more proteins can be detected on a solid support. For example, protein(s) can be attached to a solid support, the solid support can be contacted with detection agents (e.g., affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g., a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in U.S. Pat. Nos. 11,203,612 or 11,505,796 or US Pat. App. Pub. No. 2023/0167488 A1, each of which is incorporated herein by reference.

A protein can be detected based on proximity of two or more affinity agents. For example, the two affinity agents can include two components each: a receptor component and a nucleic acid component. When the affinity agents bind in proximity to each other, for example, due to ligands for the respective receptors being at the same address in an array, the nucleic acids can interact to cause a modification that is indicative of the two ligands being in proximity. Optionally, the modification can be polymerase catalyzed extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g., affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g., addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g., (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g., an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. patents application Ser. Nos. 17/062,405 and 63/159,500, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e., one and only one analyte), such as a single protein, can be individually manipulated or distinguished using a method set forth herein. A single analyte can be a single molecule (e.g., single protein), a single complex of two or more molecules (e.g., a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more analytes (e.g., an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g., in an array).

A method of the present disclosure can be performed in a multiplex format. In multiplexed configurations, different analytes can be attached to different unique identifiers (e.g., proteins can be attached to different addresses in an array). Multiplexed analytes can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 2×10⁴, 3×10⁴ or more different native-length protein primary sequences. Alternatively or additionally, a plurality of different proteins can have a complexity that is at most 3×10⁴, 2×10⁴, 1×10⁴, 1×10³, 100, 10, 5 or fewer different native-length protein primary sequences. The plurality of proteins can constitute a proteome or subfraction of a proteome. The total number of proteins that is detected, characterized or identified can differ from the number of different primary sequences in the sample from which the proteins are derived, for example, due to the presence of multiple copies of at least some protein species. Moreover, the total number of proteins that are detected, characterized or identified can differ from the number of candidate proteins suspected of being present, for example, due to the presence of multiple copies of at least some protein species, absence of some proteins in a source for the proteins, or loss of some proteins prior to analysis.

A particularly useful multiplex format uses an array of analytes (e.g., proteins) and/or affinity agents. The analytes and/or affinity agents can be attached to unique identifiers (e.g., addresses of the array) such that the analytes can be distinguished from each other. An array can be used in any of a variety of processes such as an analytical process used for detecting, identifying, characterizing or quantifying an analyte. Analytes can be attached to unique identifiers via covalent or non-covalent (e.g., ionic bond, hydrogen bond, van der Waals forces etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e., an ensemble of the analytes). Alternatively, an address can include a population of different analytes. Addresses are typically discrete in an array. Discrete addresses that neighbor each other can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

A protein or other analyte can be attached to a unique identifier (e.g., an address in an array) using any of a variety of means. The attachment can be covalent or non-covalent. Exemplary covalent attachments include chemical linkers such as those achieved using click chemistry or other linkages known in the art or described in U.S. Pat. Nos. 11,203,612 or 11,505,796 or US Pat. App. Pub. No 2023/0167488 A1, each of which is incorporated herein by reference. Non-covalent attachment can be mediated by receptor-ligand interactions (e.g., (strept) avidin-biotin, antibody-antigen, or complementary nucleic acid strands), for example, in which the receptor is attached to the unique identifier and the ligand is attached to the protein or vice versa. In particular configurations, a protein is attached to a solid support (e.g., an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful SNAPs. The use of SNAPs and other moieties to attach proteins to unique identifiers such as tags or addresses in an array are set forth in U.S. Pat. Nos. 11,203,612 or 11,505,796 or US Pat. App. Pub. No 2023/0167488 A1, each of which is incorporated herein by reference.

One or more compositions set forth herein can be present in an apparatus or vessel. For example, a composition of the present disclosure can be present in a vessel, such as a flow cell. As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g., a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

A detection apparatus can include a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in US Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534, each of which is incorporated herein by reference.

For configurations that use optical detection (e.g., luminescent detection), one or more analytes (e.g., proteins) may be immobilized on a surface, and this surface may be observed by a microscope to detect any signal from the immobilized analytes. The microscope itself may include a digital camera or other luminescence detector configured to record, store, and analyze the data collected during the scan. A luminescence detector can further include an excitation source that is capable of irradiating analytes, for example, proteins at addresses on an array, at an appropriate wavelength. A luminescence detector of the present disclosure can be configured for epiluminescent detection, total internal reflection (TIR) detection, waveguide assisted excitation, or the like. Optical filters or other optical components can be present to tune the wavelength, polarization or other optical properties of excitation and/or emission radiation used by a luminescence detector.

A light sensing device may be based upon any suitable technology, and may be, for example, a charged coupled device (CCD) sensor that generates pixilated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

A light sensing device can be configured for single molecule resolution. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features that function as addresses and facilitate single molecule resolution. For example, analytes can be distributed into wells having nanometer dimensions such as those set forth in U.S. Pat. Nos. 7,122,482 or 8,765,359, or US Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

A detection apparatus need not be configured for optical detection. For example, an electronic detector can be used for detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity reagent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

A detection apparatus can include a fluidics system, for example, configured for fluidic communication with a vessel, such as a flow cell. In some configurations, a detection apparatus can include one or more reservoirs containing affinity reagents or analytes that are delivered to a vessel. Optionally, a detection apparatus can be configured to include a waste receptacle to which waste from the vessel is collected. For example, a composition set forth herein can be delivered from the apparatus through an ingress of a flow cell and waste can be removed through an egress of the flow cell to the apparatus.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or repeating pattern of addresses. The deposition of analytes on the repeating pattern of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an array of analytes whose average spacing between analytes is relatively uniform, for example, being determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may have a random or non-repeating pattern of addresses. The deposition of analytes on the random or non-repeating pattern may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as analyte-binding sites for the coupling of analytes or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of adjacent structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A structure or feature of an array may have a characteristic dimension (e.g., a width, length, or diameter) that is smaller than a characteristic dimension of an analyte or other object (e.g., a nanoparticle) that is attached to the structure or feature. It may be preferable to provide structures or features that are smaller than analytes or other objects attached to the structure or feature to occlude the attachment of additional analytes or other objects to the structure or feature. Alternatively, a structure or feature may have a characteristic dimension that is larger than a characteristic dimension of an analyte or other object (e.g., a nanoparticle) that is attached to the structure or feature.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

An array of analytes may be provided on a solid support containing a plurality of discrete analyte-binding sites. The analyte-binding sites may be present at addresses. Each analyte-binding site may be separated from each other analyte-binding site by one or more interstitial regions. For example, each analyte-binding site may be located at a respective address, wherein the addresses are separated from each other by one or more interstitial regions. An array interstitial region may be configured to inhibit binding of analytes or other moieties to the interstitial region, for example by containing a surface coating or layer. Exemplary interstitial region surface layers or coatings can include hydrophobic moieties (e.g., hexmethyldisilazane, alkyl moieties) or hydrophilic moieties (e.g., polyethylene glycol moieties). Surface layers or coatings provided at an interstitial region can comprise linear, branched, or dendrimeric moieties. A surface layer or coating provided at an interstitial region may be a self-assembled monolayer. An address can include a single analyte-binding site (i.e. one and only one analyte-binding site or, alternatively, a plurality of analyte-binding sites can be present at a given address.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

An analyte-binding site may comprise a plurality of moieties coupled to a solid support. The plurality of moieties can include a coupling moiety and an optional plurality of passivating moieties. Preferably, a moiety containing a coupling moiety may further comprise a passivating moiety. For example, an oligonucleotide coupling moiety may further comprise a PEG passivating moiety. In some configurations, each individual moiety of a plurality of moieties coupled to an analyte-binding site can contain a coupling moiety. Alternatively, in some configurations, only a fraction of moieties of a plurality of moieties coupled to an analyte-binding site may contain a coupling moiety. Coupling moieties and passivating moieties may be provided at an analyte-binding site in a ratio of at least about 1000:1, 100:1, 10:1, 5:1, 2:1, 1:1, 1:2, 1:5, 1:10, 1:100, or 1:1000 coupling-to-passivating moieties. Alternatively or additionally, coupling moieties and passivating moieties may be provided at an analyte-binding site in a ratio of no more than about 1:1000, 1:100, 1:10, 1:5, 1:2, 1:1, 2:1, 5:1, 10:1, 100:1, or 1000:1 coupling-to-passivating moieties.

Analyte-binding sites may have an average characteristic dimension of at least about 10 nm, 25 nm, 50 nm, 100 nm, 150 nm, 200 nm, 250 nm, 300 nm, 500 nm, 1 μm, or more than 1 μm. Alternatively or additionally, analyte-binding sites may have an average characteristic dimension of no more than about 1 μm, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

An array of analytes may be provided with a characterized or characterizable analyte-binding site occupancy. The analyte-binding site occupancy can be measured as the fraction or percentage of analyte-binding sites of a plurality of analyte-binding sites containing an attached analyte. An array of analytes may be provided with an analyte-binding site occupancy of at least about 10%, 20%, 25%, 30%, 35%, 40%, 45%, 50%, 60%, 70%, 80%, 90%, 95%, 99%, 99.9%, or more than 99.9%. Alternatively or additionally, an array of analytes may be provided with an analyte-binding site occupancy of no more than about 99.9%, 99%, 95%, 90%, 80%, 70%, 60%, 50%, 45%, 40%, 35%, 30%, 25%, 20%, 10%, or less than 10%.

An array of analytes may be provided with a fraction or percentage of individual sites that each contain one and only one analyte. The fraction or percentage may be calculated relative to all other sites in the array including, but not limited to, those containing no analyte and those containing multiple analytes. Preferably, an array of analytes may be provided with super Poisson loading of single analytes (i.e., a fraction or percentage of attachments sites containing one and only one analyte exceeding 37%). An array of analytes may be provided with at least about 10%, 20%, 25%, 30%, 35%, 37%, 40%, 45%, 50%, 60%, 70%, 80%, 90%, 95%, 99%, 99.9%, or more than 99.9% of analyte-binding sites containing one and only one analyte. Alternatively or additionally, an array of analytes may be provided with no more than about 99.9%, 99%, 95%, 90%, 80%, 70%, 60%, 50%, 45%, 40%, 35%, 30%, 25%, 20%, 10%, or less than 10% of analyte-binding sites containing one and only one analyte.

It may be especially useful to provide an array of analytes with a diversity of polypeptide species. The diversity of polypeptide species may be measured with respect to a proteome, sub-proteome (e.g., a tissue proteome, a cell proteome, an organelle proteome, a metabolome, a signalome, an albuminome, etc.), or a microbiome. An array of analytes may be provided with a diversity of polypeptide species as measured by total number of polypeptide species, percentage of species of a proteome, subproteome, or microbiome, number of proteoforms of a polypeptide species, or polypeptide dynamic range.

An array of analytes may be provided with more than one unique species of polypeptide. A first polypeptide may be considered unique from a second polypeptide if the amino acid sequences of the first polypeptide and second polypeptide differ. An array of analytes may be provided with at least about 2, 5, 10, 50, 100, 500, 1000, 2000, 5000, 10000, 15000, 20000, 25000, 30000, 40000, 500000, 100000, or more than 100000 unique species of polypeptides. Alternatively or additionally, an array of analytes may be provided with no more than about 100000, 50000, 40000, 30000, 25000, 20000, 15000, 10000, 5000, 2000, 1000, 500, 100, 50, 10, 5, 2, or less than 2 unique species of polypeptides.

An array of analytes may be provided with a fraction or percentage of species of a proteome, subproteome, or microbiome. An array of analytes may be provided with at least about 0.1%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, or more than 99.9% of polypeptide species of a proteome, subproteome, or microbiome. Alternatively or additionally, an array of analytes may be provided with no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.1%, or less than 0.1% of polypeptide species of a proteome, subproteome, or microbiome.

An array of analytes may be provided with more than one proteoform of a polypeptide species. An array of analytes may be provided with more than one proteoform for two or more unique polypeptide species. Types of proteoforms of a polypeptide species can include coding variation proteoforms, translational variation proteoforms, post-translational modification proteoforms, splice variants, and combinations thereof. An array of analytes may be provided with at least about 2, 3, 4, 5, 10, 20, 50, 100, 200, 500, 1000, or more than 1000 proteoforms of a polypeptide species. Alternatively or additionally, an array of analytes may be provided with no more than about 1000, 500, 200, 100, 50, 20, 10, 5, 4, 3, or less than 3 proteoforms of a polypeptide species.

An array of analytes may be provided with a dynamic range of polypeptides. Dynamic range can refer to the ratio of abundance between a more populous polypeptide species and a less populous polypeptide species. A dynamic range can be an absolute measure (ratio of most populous polypeptide species to least populous polypeptide species) or a relative measure (ratio of a first particular polypeptide species to a second particular polypeptide species). An array of analytes may be provided with a dynamic range of at least about 10, 10², 10³, 10⁴, 10⁵, 10⁶, 10⁷, 10⁸, 10⁹, 10¹⁰, 10¹¹, 10¹², or more than 10¹². Alternatively or additionally, an array of analytes may be provided with a dynamic range of no more than about 10¹², 10¹¹, 10¹⁰, 10⁹, 10⁸, 10⁷, 10⁶, 10⁵, 10⁴, 10³, 10², 10, or less than 10.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support.

It may be useful to provide an array of analytes with one or more fiducial elements. A fiducial element may comprise a detectable address or region of the array that facilitates spatial identification on the array. A fiducial element may provide a landmark or fixed reference for determining position on an array, a measurement of length or distance on the array, a spatial reference for calibrating a detection device (e.g., a sensor or camera), and a spatial reference for registering the addresses of analyte-binding sites consistently over the timespan of an array-based process. In some cases, fiducial elements may be disposed in interstitial regions of a solid support. In other cases, fiducial elements may be disposed at analyte-binding sites of the solid support.

A fiducial element may be formed on a surface of a solid support, preferably at an interstitial region. A fiducial element can be formed by etching the surface of a solid support, thereby providing a fiducial element as a protrusion or depression of the solid support material. Alternatively, a fiducial element can be formed by depositing and/or forming a material (e.g., a metal, metal oxide, or semiconductor) on the surface of the solid support. Alternatively, a particle can be deposited on a surface of the solid support. Various lithographic techniques may be useful for providing fiducial elements on solid support surfaces at fixed addresses and with useful shapes or morphologies. A plurality of fiducial elements can be formed on a solid support at regular or patterned addresses or regions, thereby providing spatial landmarks or a scale for determining relative or absolute distance. Alternatively, fiducial elements may be formed at random locations.

Fiducial elements may be deposited at analyte-binding sites of an array of analytes. It may be preferable to deposit fiducial elements at analyte-binding sites with a random spatial distribution (e.g., providing fiducial elements at a set of analyte-binding sites having no identifiable short-range or long-range spatial pattern). A random spatial distribution of fiducial elements at analyte-binding sites may provide a spatial reference that facilitates analyte-binding site registration when detectable signals are produced from a relatively small quantity of analyte-binding sites (e.g., detectable signals produced from less than about 25%, 20%, 15%, 10%, 5%, 1%, 0.5%, or 0.1% of analyte-binding sites). It may be preferable to provide fiducial elements at at least about 0.01%, 0.05%, 0.1%, 0.5%, 1%, or more than 1% of analyte-binding sites. Alternatively or additionally, fiducial elements may be provided at no more than about 1%, 0.5%, 0.1%, 0.05%, 0.01%, or less than 0.01% of analyte-binding sites. In a preferable configuration, an analyte-binding site may contain one and only one fiducial element. In other configurations, an analyte-binding site may contain more than one fiducial element.

Fiducial elements attached to analyte-binding sites of an array of analytes may comprise a material that provides a detectable signal (e.g., a fluorescent or luminescent signal). Exemplary materials for fiducial elements can include quantum dots or fluorescently-labeled polymer particles. In some cases, a fiducial element may further comprise a coupling moiety that couples to a complementary coupling moiety that is attached to an analyte-binding site. For example, a fiducial element may be functionalized with one or more oligonucleotides that comprise a nucleotide sequence that is complementary to a nucleotide sequence of a site-coupled oligonucleotide. In some cases, a fiducial element and an analyte may be coupled to individual analyte-binding sites by the same type of coupling moiety. For example, fiducial elements and analytes may be attached to individual analyte-binding sites by hybridization of oligonucleotides on the fiducial elements or analytes to complementary oligonucleotides at analyte-binding sites. Alternatively, a fiducial element and an analyte may be coupled to individual analyte-binding sites by a different type of coupling moiety. For example, a fiducial element may be attached electrostatically to a first oligonucleotide-containing analyte-binding site while an analyte may be attached by nucleic acid hybridization to a second oligonucleotide-containing analyte-binding site

Fiducial elements may be provided at analyte-binding sites to facilitate multiplexed detection of analytes. In configurations utilizing more than one wavelength of light for optical detection, it may be preferable to provide fiducial elements that provide corresponding signals for each detected wavelength of light. For example, in a four-color detection system, it may be preferable to provide fiducial elements that provide signals for each of the four colors of light detected by the system. In some configurations, a fiducial element may be provided on an array that produces signals for each detected wavelength of light (e.g., a multi-color fluorescently-labeled polymer particle).

Fiducial elements may be deposited on an array before or after analytes are deposited on the array. A fluidic medium containing a plurality of fiducial elements may be contacted to a solid support containing a plurality of analyte-binding sites. After contacting the fluidic medium to the solid support, fiducial elements may couple to individual analyte-binding sites, preferably in a random spatial distribution. Alternatively, fiducial elements and analytes may be deposited simultaneously. A fluidic medium comprising a plurality of analyte and a plurality of fiducial elements may be contacted to a solid support containing a plurality of analyte-binding sites. After contacting the fluidic medium to the solid support, fiducial elements and analytes may couple to individual analyte-binding sites, preferably in a random spatial distribution. Additional aspects of arrays containing fiducial elements are described in U.S. Patent Publication No. 2023/0314324 A1, which is herein incorporated by reference in its entirety.

An analyte or affinity reagent can be attached to a retaining component such as a particle, array address, solid support or other substance. A particularly useful retaining component is a structured nucleic acid particle (SNAP). SNAPs can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund, Nature 440:297-302 (2006); Sigle et al, Nature Materials 20:1281-1289 (2021); or U.S. Pat. Nos. 8,501,923 or 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. Nos. 11,203,612 or 11,505,796; or US Pat. App. Pub. No. 2022/0162684 A1 or 2023/0167488 A1, each of which is incorporated herein by reference.

A retaining component, such as a SNAP, may have any of a variety of sizes and shapes to accommodate use in a desired application. For example, a retaining component can have a regular or symmetric shape or, alternatively, it can have an irregular or asymmetric shape. The shape can be rigid or pliable. The size or shape of a SNAP or other retaining component can be characterized with respect to length, area (i.e., footprint), or volume. The size or shape of a SNAP or other retaining component can be smaller than an address in an array to which it will associate or attach. Optionally, the relative sizes and shapes of an individual retaining component and an address to which it will attach are configured to preclude more than one of the retaining components from occupying the address.

Optionally, a retaining component (e.g., SNAP) or population thereof has a minimum, maximum or average length of at least about 50 nm, 100 nm, 250 nm, 500 nm, 1 micron, 5 micron or more. Alternatively or additionally, a retaining component (e.g., SNAP) or population thereof has a minimum, maximum or average length of no more than about 5 micron, 1 micron, 500 nm, 250 nm, 100 nm, 50 nm, or less.

Optionally, a retaining component (e.g., SNAP) or population thereof has a minimum, maximum or average volume of at least about 1 micron³, 10 micron³, 100 micron³, 1 mm³ or more. Alternatively or additionally, a retaining component (e.g., SNAP) or population thereof has a minimum, maximum or average volume of no more than about 1 mm³, 100 micron³, 10 micron³, 1 micron³ or less.

Optionally, the minimum, maximum or average area (i.e., footprint) for a retaining component (e.g., SNAP) is at least about 10 nm², 100 nm², 1 micron², 10 micron², 100 micron², 1 mm² or more. Alternatively or additionally, the minimum, maximum or average area for a retaining component (e.g., SNAP) footprint is at most about 1 mm², 100 micron², 10 micron², 1 micron², 100 nm², 10 nm², or less. The footprint of a retaining component (e.g., SNAP) may have a regular shape or an approximately regular shape, such as triangular, square, rectangular, circular, ovoid, or polygonal shape.

A structured nucleic acid particle (e.g., having origami or nanoball structures) may include regions of single-stranded nucleic acid, regions of double-stranded nucleic acid, or combinations thereof. For example, a SNAP can have a nucleic acid origami structure which includes a scaffold strand and a plurality of staple strands. The scaffold strand can be configured as a single, continuous strand of nucleic acid, and the staples can be formed by nucleic acid strands that hybridize, in whole or in part, with the scaffold strand.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold strand can be linear (i.e., having a 3′ end and 5′ end) or circular (i.e., closed such that the scaffold lacks a 3′ end and 5′ end). A scaffold strand can be derived from a natural source, such as a viral genome or a bacterial plasmid. For example, a nucleic acid scaffold can include a single strand of an M13 viral genome. In other configurations, a scaffold strand may be synthetic, for example, having a non-naturally occurring nucleotide sequence in full or in part. A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 2500, 5000 or more nucleotides. Alternatively or additionally, a scaffold strand may include at most about 5000, 2500, 1000, 500, 100 or fewer nucleotides.

A nucleic acid origami can include one or more oligonucleotides that are hybridized to a scaffold strand. An oligonucleotide can include two sequence regions that are hybridized to a scaffold strand, for example, to function as a ‘staple’ that restrains the structure of the scaffold. For example, a single oligonucleotide can hybridize to two regions of a scaffold strand that are separated from each other in the primary sequence of the scaffold strand. As such, the oligonucleotide can function to retain those two regions of the scaffold strand in proximity to each other or to otherwise constrain the scaffold strand to a desired conformation. Two sequence regions of an oligonucleotide staple that bind to a scaffold strand can be adjacent to each other in the nucleotide sequence of the oligonucleotide or separated by a spacer region that does not hybridize to the scaffold strand.

An oligonucleotide can include a first sequence region that is hybridized to a complementary sequence of a nucleic acid origami and a second region that provides a “handle” or “linker” for attaching another moiety. For example, the moiety can include an analyte (e.g., protein), paratope, affinity moiety (e.g., antibody), organic linker, inorganic ion, docker or tether. Optionally, the moiety can be attached to an oligonucleotide that is complementary to the second region of the handle and the moiety can be attached to the nucleic acid origami via hybridization of the handle to the complementary oligonucleotide.

Oligonucleotides can be configured to hybridize with a nucleic acid scaffold, another oligonucleotide, a staple oligonucleotide, or a combination thereof. One or more regions of an oligonucleotide that hybridizes to another sequence of a nucleic acid origami or other structured nucleic acid particle can be located at or near 5′ end of the oligonucleotide, at or near 3′ end of the oligonucleotide, or in a region of the oligonucleotide that is between the end regions. The oligonucleotides can be linear (i.e., having a 3′ end and a 5′ end) or closed (i.e. circular, lacking both 3′ and 5′ ends). An oligonucleotide that is included in a nucleic acid origami or other structured nucleic acid particle can have any of a variety of lengths including, for example, at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide may form a hybrid of at least about 5, 6, 7, 8, 9, 10, 15, 20, 25, 50 or more consecutive or total base pairs with another nucleotide sequence of a nucleic acid origami. Alternatively or additionally, an oligonucleotide may form a hybrid of no more than about 50, 25, 20, 15, 10, 9, 8, 7, 6, 5, or fewer consecutive or total base pairs with another nucleotide sequence.

A retaining component may be provided with moieties that facilitate a binding interaction with a surface of a solid support, or moieties coupled to the surface of the solid support. Moieties that facilitate coupling of a retaining component to a solid support may be configured to form a covalent interaction or a non-covalent interaction with the solid support or a moiety coupled to the solid support. In an example, a retaining component may be provided with one or more nucleic acid strands that can hybridize to a complementary nucleic acid strand on a surface of a solid support by nucleic acid hybridization. Preferably, a retaining component may be provided with a plurality of moieties that can bind to a surface of a solid support. In some cases, the moieties may be pendant from the retaining component. Pendant moieties may include a linking moiety that increases the length of the moiety and/or increases the flexibility or spatial degrees of freedom of the moiety. A linking moiety can be, for example, a single-stranded nucleic acid (e.g., with a nucleotide sequence that is not complementary to a surface-bound oligonucleotide), a peptide linker, or a synthetic polymer (e.g., polyethylene glycol, alkyl moieties, etc.).

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. Nos. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Other useful retaining components include artificial polymers. Artificial polymers can include polymers that are made by human activity rather than occurring naturally. For example, a polymer that is made at least in part by human activity or that includes at least one artificial moiety is referred to as an “artificial polymer.” In some cases, the artificial polymers are configured as dendrons. A dendron will include at least one branched chain polymer. A branched chain polymer can include at least 1, 2, 3, 4, 5, 6, 8 or 10 branch points. Alternatively or additionally, a branched chain can include at most 10, 8, 6, 5, 4, 3, 2 or 1 branch points. A branch point is a covalent intersection between at least two chains. For example, at least 2, 3, 4, 5 or more chains can intersect at a branch point of a branched chain. Alternatively or additionally, at most 5, 4, 3 or 2 chains can intersect at a branch point of a branched chain. A polymer, whether branched or not, can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

An retaining component that includes an artificial polymer can have a length, volume or footprint in a range set forth above. A retaining component can be further characterized in terms of molecular weight (or molecular weight distribution) in a desired size range. For example, the molecular weight, average molecular weight distribution, minimum molecular weight distribution or maximum molecular weight distribution can be at least 1 kDa, 2 kDa, 5 kDa, 10 kDa, 25 kDa, 50 kDa or more. Alternatively or additionally, the molecular weight, average molecular weight distribution, minimum molecular weight distribution or maximum molecular weight distribution can be at most 50 kDa, 25 kDa, 10 kDa, 5 kDa, 2 kDa, 1 kDa or less. A retaining component can be characterized in terms of radius of gyration. For example, the radius of gyration can be at least about 2 nm, 5 nm, 10 nm, 15 nm, 25 nm, 50 nm or more. Alternatively or additionally, retaining component can be configured to have a radius of gyration that is at most about 50 nm, 25 nm, 15 nm, 10 nm, 5 nm, 2 nm or less. An artificial polymer can be characterized in term of degree of polymerization (i.e. number of monomer subunits) present. For example, an artificial polymer can include at least 2, 10, 20, 30, 40, 50, 100, 200, 300 or more monomers. Alternatively or additionally, an artificial polymer can include at most 300, 200, 100, 50, 40, 30, 20, 10, or 2 monomers.

An artificial polymer can lack natural polymers or monomers found in natural polymers. For example, the skeletal structure of the artificial polymer can lack natural polymers or monomers. This can be the case whether or not the artificial polymer has attached moieties that include natural polymers or monomers. Examples of natural moieties that can be absent from an artificial polymer, for example in the skeletal structure include, but are not limited to, nucleic acids (e.g., DNA or RNA), nucleotides (e.g., deoxyribonucleotides or ribonucleotides), nucleosides (e.g., deoxyribonucleosides or ribonucleosides), peptides (e.g., proteins, polypeptides or oligopeptides), amino acids, or sugars (e.g., saccharide monomers, monosaccharides, oligosaccharides, polysaccharides or glycans). An artificial polymer can optionally lack any polymer or monomer that is synthesized in vivo or that is capable of being synthesized in vivo. Alternatively, an artificial polymer can include natural moieties that are combined to form a non-naturally occurring molecule. For example, an artificial polymer can be composed of nucleic acid monomers or nucleic acid strands that form a non-naturally occurring nucleic acid dendrimer structure.

Particularly useful artificial polymers include, for example, poly (amidoamine) (PAMAM) dendrimer, poly (amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Examples of useful polymers are set forth in Tomalia, et al. J Polym Sci Part A: Polym Chem 40:2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25 (17): 3982 (2020); Zheng, et al. or Chem. Soc. Rev. 44, 4091-4130 (2015), each of which is incorporated herein by reference.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-Sdy Tag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 or 11,505,796, each of which is herein incorporated by reference in its entirety.

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both time-scale and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems and methods utilizing arrays, as set forth herein, may have binding interactions that are unwanted, unexpected, or contrary-to-design. The binding interactions (e.g., covalent or non-covalent) can occur between an array surface or array feature and an unbound moiety that may become contacted with the array surface or array feature. Such interactions may be referred to hereinafter as “orthogonal binding phenomena.” Orthogonal binding phenomena may be qualitatively characterized as a binding interaction that occurs in a system that is expected or engineered to prevent such a binding interaction (e.g., a putatively hydrophilic molecule binding to a putatively hydrophobic surface). Orthogonal binding phenomena may be quantitatively characterized as measurable binding interactions occurring between an array surface or array feature (e.g., an interstitial region, an analyte address) and moiety that may transition from a fluid-phase state to an immobilized state, for example, by attaching to the array surface or feature, wherein the measurable binding interactions occur at a rate and/or to an extent that exceeds a predicted rate and/or extent. For example, the rate or extent may exceed a thermodynamic or kinetic prediction (e.g., a dissociation constant, a binding on-rate, a binding off-rate, etc.). By way of further example, if an unbound moiety is characterized to bind to a surface-coupled passivating moiety (e.g., polyethylene glycol) with a kilomolar dissociation constant (a very weak binding interaction), then observing a millimolar binding dissociation constant between the unbound moiety and an array surface that is provided with a uniform layer of the surface-coupled passivating moiety would indicate an orthogonal binding phenomena (i.e., binding due to a mechanism other than the specific binding of the unbound moiety to the surface-coupling passivating moiety). Orthogonal binding phenomena may be characterized based upon a stochastic measure, such as spatial and/or temporal variations in unwanted, unexpected, or contrary-to-design binding phenomena.

The presence, type or degree of orthogonal binding phenomenon is typically contextual, for example, relating to the conditions in which a binding interaction occurs. Consider, for example, a surface that is engineered to comprise a material that prevents binding of an assay reagent to the surface but also contains an unwanted impurity or a vacancy that can form a binding interaction with the assay reagent. Independent of any predictability or specificity that may be ascribed to the binding interaction between the impurity and the assay reagent, it is unwanted and unintended. Accordingly, “non-orthogonal” and “orthogonal” binding interactions can be discerned in the context of the intended use of the system within which they occur. In the context of a molecular array, such as a single-analyte array, the molecular array may be engineered to specifically retain chosen moieties at particular sites and inhibit binding of particular moieties at other sites or regions. In such a context, orthogonal binding interactions may include binding of moieties other than the chosen moieties at the particular sites, as well as binding of particular moieties at the other sites or regions.

In some configurations of a method or apparatus set forth herein, an assay reagent may recognize or bind an analyte that is a target analyte for the assay reagent. Nevertheless, the assay reagent may orthogonally bind to non-target materials or substances, such as non-target materials or substances present in an array that also includes the target analyte (e.g., anchoring moieties). Accordingly, orthogonal binding phenomena may be defined in certain cases as “analyte orthogonal binding interactions” or “non-analyte orthogonal binding interactions.” An analyte orthogonal binding interaction may refer to a binding interaction between a moiety and a non-analyte component of a single-analyte system, in which the moiety is expected or intended to form a binding interaction with an analyte of a single-analyte system. For example, an analyte orthogonal binding interaction may comprise an affinity agent becoming bound to an interstitial region of a single-analyte array. A non-analyte binding interaction may refer to a binding interaction between a moiety and a component of a single-analyte system (including an analyte), in which the moiety is not expected or intended to form a binding interaction with the component of a single-analyte system. For example, a blocking moiety (e.g., albumin, dextran, etc.) that is configured to remain unbound in solution may become bound to a surface-coupled molecule adjacent an analyte on a single-analyte array, thereby sterically impeding access to the analyte.

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

A fluidic medium may be formulated with any one of numerous components depending upon its intended application. A fluidic medium can comprise one or more solvents. A single-phase fluidic medium can comprise two or more miscible solvents. In a mixture of miscible solvents, a solvent may be considered a base solvent if it comprises a greater than 50% fraction on a mass, molar, or volumetric basis. A miscible solvent may be mixed into a base solvent to alter a physical property of the base solvent, such as polarity, density, pH, viscosity, or surface tension. A fluidic medium can comprise a polar solvent or a non-polar solvent. A fluidic medium can comprise a protic or aprotic solvent. A fluidic medium can comprise an aqueous medium. A fluidic medium can comprise an organic solvent, such as acetic acid, acetone, acetonitrile, benzene, a butanol, 2-butanone, carbon tetrachloride, chlorobenzene, chloroform, cyclohexane, 1,2-dichloroethane, diethylene glycol, diethyl ether, diglyme, 1,2-dimethoxy-ethane, dimethylformamide, dimethyl sulfoxide, 1,4-dioxane, ethanol, ethyl acetate, ethylene glycol, glycerin, heptane, hexamethylphosphoramide, hexamethylphophorus triamide, hexanes, methanol, methyl t-butyl ether, methylene chloride, N-methyl-pyrrolidinone, nitromethane, pentane, petroleum ether, 1-proponal, 2-propanol, pyridine, tetrahydrofuran, toluene, triethyl amine, xylene, or a combination thereof. A fluidic medium can comprise a polar solvent, such as N-methyl pyrrolidone, tetrahydrofuran, ethyl acetate, acetone, dimethylfuran, acetonitrile, dimethyl sulfoxide, propylene carbonate, N-butanol, isopropyl alcohol, nitromethane, ethanol, methanol, acetic acid, or a combination thereof. A fluidic medium can comprise a non-polar solvent, such as benzene, carbon tetrachloride, chloroform, cyclohexane, dichloromethane, dimethoxyethane, ethyl ether, heptane, hexachloroethane, hexane, limonene, naphtha, pentane, tetrachloroethylene, tetrahydrofuran, toluene, xylenes, and combinations thereof. In some cases, a fluidic medium may comprise an aprotic solvent, such as N-methyl pyrrolidone, tetrahydrofuran, ethyl acetate, acetone, dimethylfuran, acetonitrile, dimethyl sulfoxide, propylene carbonate, or a combination thereof.

A fluidic medium may further comprise one or more components, including: 1) an ionic species, 2) a buffering agent, 3) a surfactant or detergent, 4) a chelating agent, 5) a denaturing agent or a chaotrope, 6) a cosmotropic or crowding agent, 7) a clouding agent, 8) a reactive scavenger, and 9) a blocking agent.

A fluidic medium may comprise one or more ionic species. An ionic species may be provided to a fluidic medium as a salt, thereby providing an anionic species and a cationic species to the fluidic medium. An ionic species can include a zwitterionic species. A fluidic medium may comprise a cationic species such as Na⁺, K⁺, Ag⁺, Cu⁺, NH₄⁺, Mg²⁺, Ca²⁺, Cu²⁺, Cd²⁺, Zn²⁺, Fe²⁺, Co²⁺, Ni²⁺, Cr²⁺, Mn²⁺, Ge²⁺, Sn²⁺, Al³⁺, Cr³⁺, Fe³⁺, Co³⁺, Ni³⁺, Ti³⁺, Mn³⁺, Si⁴⁺, V⁴⁺, Ti⁴⁺, Mn⁴⁺, Ge⁴⁺, Se⁴⁺, V⁵⁺, Mn⁵⁺, Mn⁶⁺, Se⁶⁺, and combinations thereof. A fluidic medium may comprise an anionic species such as F⁻, Cl⁻, Br⁻, ClO₃⁻, H₂PO₄⁻, HCO₃⁻, HSO₄⁻, OH⁻, I⁻, NO₃⁻, NO₂⁻, MnO₄⁺, SCN⁻, CO₃²⁻, CrO₄²⁻, Cr₂O₇²⁻, HPO₄²⁻, SO₄²⁻, SO₃²⁻, PO₄³⁻, and combinations thereof. A fluidic medium may comprise a chelating agent, such as ethylenediaminetetraacetic acid (EDTA), nitrilotriacetic acid, n-hydroxyethylenediaminetetraacetic acid (HEDTA), oxalic acid, malic, acid, rubeanic acid, citric acid, or combinations thereof.

A fluidic medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, PBS, and CABS.

A fluidic medium may comprise a surfactant or detergent. A surfactant or detergent may comprise a cationic surfactant or detergent, an anionic surfactant or detergent, a zwitterionic surfactant or detergent, an amphoteric surfactant or detergent, or a non-ionic surfactant or detergent. A fluidic medium may include a surfactant species including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentaethylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-β-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio) butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, N-laurylsarcosine, and combinations thereof.

A fluidic medium may comprise a denaturing or chaotropic species, such as acetic acid, trichloroacetic acid, sulfosalicylic acid, sodium bicarbonate, ethanol, ethylenediamine tetraacetic acid (EDTA), urea, guanidinium chloride, lithium perchlorate, sodium dodecyl sulfate, 2-mercaptoethanol, dithiothreitol, tris (2-carboxyethyl) phosphine (TCEP), or a combination thereof. A denaturing or chaotropic species may be provided to alter a conformational state of an array component (e.g., causing denaturation of a polypeptide), or may be provided to maintain a conformational state of an array component (e.g., maintaining a polypeptide in a denatured or partially-denatured state).

A fluidic medium may comprise a cosmotropic species, such as carbonate ion, sulfate ion, phosphate ion, magnesium ion, lithium ion, zinc ion, aluminum ion, trehalose, glucose, proline, tert-butanol, or a combination thereof. A fluidic medium may comprise a clouding agent such as sodium chloride, potassium chloride, sodium bromide, potassium bromide, sodium nitrate, sodium sulfate, sodium phosphate, or a combination thereof. A cosmotropic species may be provided to decrease a separation distance between molecules and array components (e.g., causing smaller separation between an affinity agent and an analyte).

A fluidic medium may comprise a reactive scavenger species. A reactive scavenger may be provided to reduce solution-phase concentrations of reactive species (e.g., oxidizing or reducing species). A reactive scavenger may be provided during a photon-mediated process (e.g., fluorescent imaging) to reduce photodamage or other deleterious photon-related processes (e.g., singlet oxygen generation, free radical generation). Exemplary reactive scavenger species can include ascorbic acid, 9,10-anthracenediyl-bis (methylene) dimalonic acid (ABDA), epigallocatechin gallate (EPGG), N-acetyl-L-cysteine, caffeic acid, reseveratrol, 4-hydroxy-2,2,6,6-tetramethylpiperidin-1-oxyl (TEMPOL), sodium sulfite, 1,4-diazabicyclo[2.2.2] octane (DABCO), sodium pyruvate, N,N′-dimethylthiourea (DMTU), mannitol, dimethyl sulfoxide (DMSO), 6-hydroxy-2,5,7,8-tetramethylchroman-2-carboxylic acid (Trolox), 2-phenyl-1,2-benzisoselenazol-3(2H)-one (Ebselen), α-tocopherol, uric acid, sodium azide, manganese (III)-tetrakis (4-benzoic acid) porphyrin, 4,5-dihydroxybenzene-1,3-disulfonate, or a combination thereof. Other useful reactive scavengers and methods for their use in reducing photodamage or other deleterious photon-related processes are set forth in U.S. Pat. No. 10,106,851, which is incorporated herein by reference.

A fluidic medium may comprise a blocking agent. A blocking agent may include any species that inhibits orthogonal binding phenomena between assay agents and array components, such as polyethylene glycol, dextrans, albumin, or synthetic polymers such as PF-127 or polyvinylpyrrolidone.

A method set forth herein may involve a step of delivering a fluidic medium to a vessel (e.g., a flow cell, a fluidic cartridge, a reactor or microreactor, etc.) containing an array, as set forth herein. In some cases, after delivering a fluidic medium to a vessel, the fluidic medium may be incubated with an array within the vessel. Incubation of a fluidic medium with an array may be substantially quiescent. Alternatively, incubation of a fluidic medium with an array may be non-quiescent due to mixing, agitation, or circulation of the fluidic medium within or through the vessel.

A method set forth herein may involve a step of altering a fluidic medium with respect to one or more properties of the fluidic medium. Altered properties can include temperature, pH, ionic strength, and composition of the fluidic medium. In some cases, altering a fluidic medium may comprise displacing a first fluidic medium having a first property (e.g., temperature, pH, ionic strength, composition) with a second fluidic medium having a second property, in which the first property differs from the second property. In other cases, altering a fluidic medium may comprise mixing a second fluidic medium or chemical component (e.g., a solute) into a first fluidic medium. For example, a pH of a fluidic medium may be altered by adding an acid or base species to a fluidic medium in a vessel. In another example, a fluidic medium may be diluted or condensed with respect to ionic strength or concentration of a component by addition of a second fluidic medium to the vessel.

A fluidic medium may be provided at, heated to, cooled to, or maintained at a temperature of at least about −80 degrees Celsius (° C.), −50° C., −10° C., −5° C., 0° C., 5° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 60° C., 70° C., 80° C., 90° C., 95° C., or more than 95° C. Alternatively or additionally, a fluidic medium may be provided at, heated to, cooled to, or maintained at a temperature of no more than about 95° C., 90° C., 80° C., 70° C., 60° C., 50° C., 45° C., 40° C., 35° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 5° C., 0° C., −5° C., −10° C., −50° C., −80° C., or less than −80° C. A temperature of a fluidic medium may be adjusted to a value set forth herein in order to alter a conformation of a macromolecule. A temperature of a fluidic medium may be adjusted to a value set forth herein in order to dissociate a binding interaction set forth herein.

A fluidic medium may be provided at or adjusted to a pH of at least about 0.0, 0.5, 1.0, 1.5, 2.0, 2.5, 3.0, 3.5, 4.0, 4.5, 5.0, 5.5, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.5, 9.0, 9.5, 10.0, 10.5, 11.0, 11.5, 12.0, 12.5, 13.0, 13.5, 14.0, or more than 14.0. Alternatively or additionally, a fluidic medium may be provided at or adjusted to a pH of no more than about 14.0, 13.5, 13.0, 12.5, 12.0, 11.5, 11.0, 10.5, 10.0, 9.5, 9.0, 8.5, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.5, 5.0, 4.5, 4.0, 3.5, 3.0, 2.5, 2.0, 1.5, 1.0, 0.5, or less than 0.5. A pH of a fluidic medium may be adjusted to a value set forth herein in order to alter a conformation of a macromolecule. A pH of a fluidic medium may be adjusted to a value set forth herein in order to dissociate a binding interaction set forth herein.

A component of a fluidic medium may be provided at or adjusted to a molar concentration of at least about 0.0001 moles per liter (M), 0.001M, 0.01M, 0.02M, 0.03M, 0.04M, 0.05M, 0.06M, 0.07M, 0.08M, 0.09M, 0.1M, 0.2M, 0.3M, 0.4M, 0.5M, 0.6M, 0.7M, 0.8M, 0.9M, 1M, 1.1M, 1.2M, 1.3M, 1.4M, 1.5M, 1.6M, 1.7M, 1.8M, 1.9M, 2M, 2.1M, 2.2M, 2.3M, 2.4M, 2.5M, 2.6M, 2.7M, 2.8M, 2.9M, 3M, 3.1M, 3.2M, 3.3M, 3.4M, 3.5M, 3.6M, 3.7M, 3.8M, 3.9M, 4M, 4.1M, 4.2M, 4.3M, 4.4M, 4.5M, 4.6M, 4.7M, 4.8M, 4.9M, 5M, 5.1M, 5.2M, 5.3M, 5.4M, 5.5M, 5.6M, 5.7M, 5.8M, 5.9M, 6M, 7M, 8M, 9M or more than 10M. Alternatively or additionally, a component of a fluidic medium may be provided at or adjusted to a molar concentration of no more than about 10 M, 9M, 8M, 7M, 6M, 5.9M, 5.8M, 5.7M, 5.6M, 5.5M, 5.4M, 5.3M, 5.2M, 5.1M, 5.0M, 4.9M, 4.8M, 4.7M, 4.6M, 4.5M, 4.4M, 4.3M, 4.2M, 4.1M, 4.0M, 3.9M, 3.8M, 3.7M, 3.6M, 3.5M, 3.4M, 3.3M, 3.2M, 3.1M, 3.0M, 2.9M, 2.8M, 2.7M, 2.6M, 2.5M, 2.4M, 2.3M, 2.2M, 2.1M, 2.0M, 1.9M, 1.8M, 1.7M, 1.6M, 1.5M, 1.4M, 1.3M, 1.2M, 1.1M, 1.0M, 0.9M, 0.8M, 0.7M, 0.6M, 0.5M, 0.4M, 0.3M, 0.2M, 0.1M, 0.09M, 0.08M, 0.07M, 0.06M, 0.05M, 0.04M, 0.03M, 0.02M, 0.01M, 0.001M, 0.001M, or less than about 0.001M. A molar concentration of a fluidic medium may be adjusted to a value set forth herein in order to alter a conformation of a macromolecule. A molar concentration of a fluidic medium may be adjusted to a value set forth herein in order to dissociate a binding interaction set forth herein.

A component of a fluidic medium may be provided at or adjusted to a weight or volumetric percentage of at least about 0.0001%, 0.001%, 0.005%, 0.01%, 0.05%, 0.1%, 0.5%, 1%, 2%, 3%, 4%, 45%, 6%, 7%, 8%, 9%, 10%, 20%, 30%, 40%, 50%, or more than 50%. Alternatively or additionally, a component of a fluidic medium may be provided at or adjusted to a weight or volumetric percentage of no more than about 50%, 40%, 30%, 20%, 10%, 9%, 8%, 7%, 6%, 5%, 4%, 3%, 2%, 1%, 0.5%, 0.1%, 0.05%, 0.01%, 0.005%, 0.001%, 0.0001%, or less than 0.0001%. A weight or volumetric percentage of a fluidic medium may be adjusted to a value set forth herein in order to alter a conformation of a macromolecule. A weight or volumetric percentage of a fluidic medium may be adjusted to a value set forth herein in order to dissociate a binding interaction set forth herein.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g., organelles). For example, a sample can be derived from a tissue biopsy, biological fluid (e.g., blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g., fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g., an individual human), single tissue, single cell, single organelle (e.g., endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 μg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g., cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g., shorter or longer amino acid sequences), different arrangement of domains (e.g., transcriptional splice variants), or different post translational modifications (e.g., presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglycylation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g., sequence agnostic) phosphatase such as alkaline phosphatase can be useful. Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PHP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, at least eighteen different protein lysine deacetylases (e.g., histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylgucosaminidase can be used to cleave non-reducing terminal β-linked N-acetylgucosamine from proteins; endo-a-N-acetylgalactosaminidase can be used to remove O-glycosylation, for example, removing serine- or threonine-linked unsubstituted Galb1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

In addition to the foregoing reagents, also provided herein are kits useful in carrying out the analyses described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the flow-cells and arrays used to immobilize proteins of interest in a single molecule, in an optically detectable format for subsequent analysis in appropriately configured optical detection systems described herein. Such kits can include instructions for carrying out the enrichment, flow-cell deposition, interrogation and follow on analysis of biological samples using such kits.

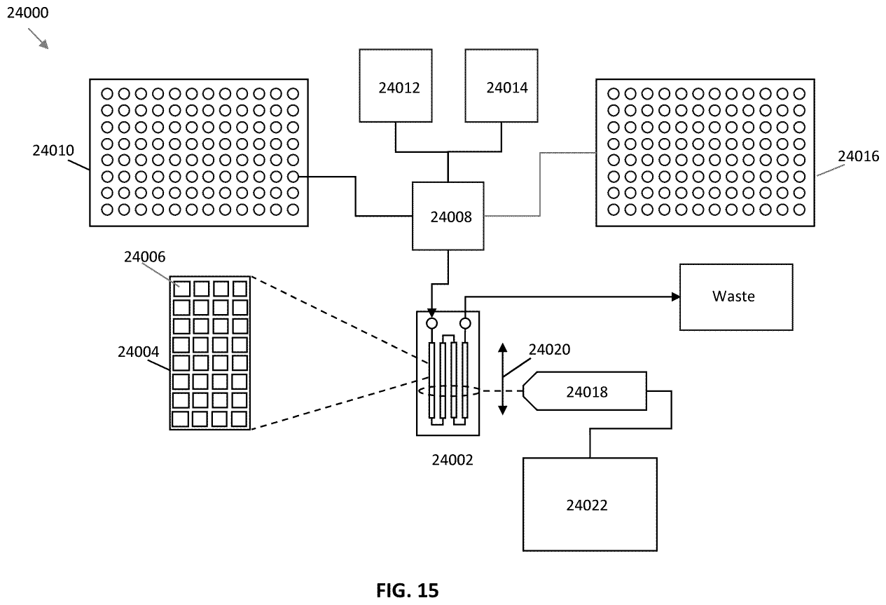

Additionally, provided herein are systems for performing the techniques, reagents, systems, and methods described herein. An example of a system is illustrated in FIG. 15. As shown, the system 24000 includes a flowcell 24002 that includes an array surface (shown as 24004) within the channels of the flow cell upon which individual protein molecules from a sample may be deposited and immobilized in locations 24006 that are individually addressable, and in particular cases are individually optically resolvable from each other using, e.g., fluorescence microscopy or scanning techniques.

The system will also typically include a fluidic delivery system 24008 that is configured to deliver different fluids to the flow cell 24002 through a series of fluidic lines and utilizing appropriate pumps, valves and other conventional fluid controls. The fluidics system 24008 may be fluidically coupled to various sources of fluids and reagents needed to carry out the analysis on the flow cell. For example, as shown, fluidic system 24008 is fluidly coupled to a source of a plurality of reagents 24010 (shown as a 96 well plate, although any number of different reagent storage systems of varying capacity may be employed) that includes a library of multiple affinity reagents that each have affinity for different characteristics of one or more proteins of interest. Additionally, fluidic system 24008 may also be coupled to sources of washing fluids or buffers 24012, and removal reagents 24014 (for removing bound affinity reagents following detection), as well as any other ancillary fluids and reagents needed for the analysis. Similarly, where flow cells are prepared on the system, the fluidic system may be coupled to sources of different sample materials that are to be analyzed 24016 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically includes a detection system, such as optical detection system 24018, for detecting and recording fluorescent signals arising from different positions on the array surface. Such detection systems may generally include line scanning confocal fluorescent microscope systems, which are capable of scanning across large array surfaces (as shown by arrow 24020) to detect and record fluorescence across such surfaces at reasonably high scan rates.

The overall systems also typically include one or more computers or processors 24022 for controlling the operation of the instrument system including the fluidic system 24008 (e.g., to sample different sample sources 24016, reagent sources 2010 and delivery timing and volume of each), and detection system 24018, among other functions, and for recording the detected signals received from the detection system 24018, e.g., fluorescent signals, and analyzing such signals to identify potential binding by each of the different affinity reagents. Processors 24022 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been describe in, for example, U.S. Pat. Nos. 11,545,234, 10,473,654B1, and Eggertson, et al., A theoretical framework for proteome-scale single-molecule protein identification using multi-affinity protein binding reagents, bioRxiv, https://doi.org/10.1101/2021.10.11.463967, U.S. Patent Application No. 2022/0236282, International Patent Application Nos. PCT/US24/15132, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 24022 can be an electronic device of a detection system, the electronic device being integral to the detection system or remotely located with respect to the detection system. The computer system 24022 includes a computer processing unit (CPU, also “processor” and “computer processor” herein), which can be a single core or multi core processor, or a plurality of processors for parallel processing. The computer system 24022 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 24022 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 24022, can implement a peer-to-peer network, which may enable devices coupled to the computer system 24022 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 24022 can be included in the circuit. In some cases, the circuit is an application specific integrated circuit (ASIC).

The storage unit can store files, such as drivers, libraries and saved programs. The storage unit can store user data, e.g., user preferences and user programs. The computer system 24022 in some cases can include one or more additional data storage units that are external to the computer system 24022, such as located on a remote server that is in communication with the computer system 24022 through an intranet or the Internet.

The computer system 24022 can communicate with one or more remote computer systems through the network. For instance, the computer system 24022 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 24022 via the network.

Methods as described herein can be implemented by way of machine (e.g., computer processor) executable code stored on an electronic storage location of the computer system 24022, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 24022, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 24022 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

1. A composition, comprising:
a) a particle comprising a first face and a second face, wherein the first face is substantially opposed to the second face, wherein the second face comprises a first attachment site containing a first attachment moiety, and wherein the second face further comprises a second attachment site containing a second attachment moiety;
b) a plurality of coupling moieties coupled to the first face; and
c) an analyte, wherein the analyte comprises a first complementary attachment moiety and a second complementary attachment moiety, wherein the first complementary attachment moiety is attached to the first attachment moiety, and wherein the second complementary attachment moiety is attached to the second attachment moiety.

2. The composition of claim 1, wherein the second face further comprises a third attachment site, wherein the third attachment site contains a third attachment moiety.

3.-6. (canceled)

7. The composition of claim 1, wherein the first complementary attachment moiety is covalently attached to the first attachment moiety.

8.-9. (canceled)

10. The composition of claim 1, wherein the analyte comprises a polymeric chain.

11. (canceled)

12. The composition of claim 10, wherein the polymeric chain comprises a covalently linked sequence of residues, wherein a first residue of the sequence of residues comprises the first complementary attachment moiety, and wherein the second residue of the sequence of residues comprises the second complementary attachment moiety.

13.-15. (canceled)

16. The composition of claim 1, wherein the second face of the particle has a maximum length of at least 50 nanometers (nm).

17.-18. (canceled)

19. The composition of claim 1, wherein the second face of the particle comprises a spacing moiety, wherein the spacing moiety attaches the first attachment moiety or the second attachment moiety to the second face.

20.-25. (canceled)

26. The composition of claim 1, wherein the second face further comprises an avidity component.

27.-32. (canceled)

33. The composition of claim 1, further comprising a detectable probe.

34.-39. (canceled)

40. The composition of claim 1, wherein the second attachment moiety comprises an analyte-binding group.

41.-44. (canceled)

45. A method, comprising:
a) contacting an analyte to a particle, wherein the particle comprises a first attachment site comprising a first attachment moiety, and a second attachment site comprising a second attachment moiety;
b) attaching a first complementary attachment moiety of the analyte to the first attachment moiety of the particle, and attaching a second complementary attachment moiety of the analyte to the second attachment moiety of the particle; and
c) coupling the particle to a site of a solid support.

46.-47. (canceled)

48. The method of claim 45, further comprising forming the first complementary attachment moiety on the analyte.

49.-50. (canceled)

51. The method of claim 45, wherein attaching the first complementary attachment moiety of the analyte to the first attachment moiety of the particle comprises hybridizing a nucleic acid of the first complementary attachment moiety to a complementary nucleic acid of the first attachment moiety.

52. (canceled)

53. The method of claim 45, wherein attaching the second complementary attachment moiety of the analyte to the second attachment moiety of the particle comprises coupling an analyte-binding group of the second attachment moiety to the analyte.

54.-55. (canceled)

56. The method of claim 53, further comprising, after coupling the analyte-binding group of the second attachment moiety to the analyte, attaching a third complementary attachment moiety of the analyte to a third attachment moiety at a third attachment site of the particle.

57. The method of claim 53, further comprising, after coupling the analyte-binding group of the second attachment moiety to the analyte, attaching a third complementary attachment moiety of the analyte to a third attachment moiety at the second attachment site of the particle.

58. The method of claim 45, further comprising, after coupling the particle to the solid support, contacting a sensing particle comprising a detectable label to the solid support, wherein the sensing particle further comprises a moiety that is complementary to a third attachment moiety of a third attachment site.

59. The method of claim 58, further comprising detecting presence or absence of a signal from the detectable label at an address of the solid support containing the particle.

60.-71. (canceled)

72. A composition comprising:
a first structure having a first surface with an attachment moiety;
a particle having a first surface and a second surface, wherein the first surface and the second surface are opposed to each other, and wherein the first surface is attached with the first surface of the first structure;
a macromolecule attached with the second surface of the particle; and
a second structure having a first surface, wherein the first surface of the second structure is attached with the macromolecule.

73.-74. (canceled)

75. A method comprising:
contacting an analyte to a particle to form an analyte-particle complex;
contacting the analyte-particle complex to a first structure on a surface, wherein the analyte-particle complex is positioned between the first structure and a second structure on the surface, and wherein the analyte of the analyte-particle complex is positioned distal to the first structure in comparison to the particle of the analyte-particle complex;
denaturing the analyte of the analyte-particle complex to form a denatured analyte-particle complex; and
attaching the analyte of the denatured analyte-particle complex to the second structure.

76.-84. (canceled)
